(19) United States
(12) Patent Application Publication
(10) Pub. No.: US 2011/0275563 A1
(43) Pub. Date: Nov. 10, 2011

(54) Use of CTGF as a Cardioprotectant

(76) Inventors: Havard Attramadal, Oslo (NO); M. Shakil Ahmed, Oslo (NO); Vladimir N. Martinov, Oslo (NO); Guro Ellen Valen, Jar (NO); Jørgen Gravning, Oslo (NO); Ingvild Trondstad Moe, Oslo (NO)

(21) Appl. No.: 13/063,860

(22) PCT Filed: Sep. 17, 2009
(86) PCT No.: PCT/GB09/02218

§ 371 Date: Jul. 29, 2011

(30) Foreign Application Priority Data
Sep. 18, 2008 (GB) 0817148.0
Nov. 28, 2008 (GB) 0821800.0

(51) Int. Cl. A61K 38/18; A61P 9/00; C07K 14/475

(52) U.S. Cl. 514/7.6; 530/399

(57) ABSTRACT

The present invention is based upon the surprising discovery that Connective Tissue Growth Factor (CTGF) may act directly on the heart as a cardioprotective factor and provides CTGF for use in the treatment of a subject who has incurred or is incurring damage to the heart, wherein said CTGF is for administration during or after the heart-damaging event. The invention also relates to CTGF for use in protection of the heart during or after surgery or during ex vivo transportation or storage of an explanted heart.

The present invention is based upon the surprising discovery that Connective Tissue Growth Factor (CTGF) may act directly on the heart as a cardioprotective factor, both to protect against and to treat or alleviate damage to the heart, specifically damage caused by injury or disease. CTGF may be used as a therapeutic or prophylactic agent for the treatment or prevention of damage to the heart due to injury or disease. However, more particularly, the CTGF may be used according to the present invention in the treatment of damage to the heart that has already occurred or is occurring, i.e. during or after a heart-damaging event. Thus, CTGF may be used in the treatment of acute damage to the heart, or in another aspect, in the acute treatment of heart damage, more precisely myocardial damage. Beneficially, CTGF may be used in the treatment of a subject incurring or who has incurred heart damage, to prevent, reduce or minimise further damage to the heart, or the development of heart disease subsequent to (or as a result from) the initial heart damaging event.

CTGF may thus be used to treat or to prevent, or to delay or reduce the development of, heart conditions such as acute coronary syndromes, including myocardial infarction (MI) and angina, heart failure and conditions which predispose to, or lead to the development of heart failure, for example ischaemic heart disease, left ventricular dysfunction, left ventricular remodelling and cardiomyopathy, as well as to protect the heart from damage during or after surgery, including during ex vivo transportation of an explanted heart.

Cardiovascular disease, and in particular heart disease, is a major cause of death and morbidity worldwide. Such diseases thus have a vast socio-economic impact, both in terms of health-care costs and in productivity lost due to death and disability.

Heart disease, which includes any disease or disorder or condition of the heart which is generally characterised by impaired cardiac function, e.g. heart failure, affects a large number of people throughout the world, and in particular the Western world. It is responsible for a reduced quality of life and premature death in a significant proportion of sufferers and may occur in men, women and children of both sexes, but is particularly prevalent in men and in the elderly or middle-aged.

The specific condition heart failure is characterized by impaired cardiac function, in terms of the ability of the heart to provide the tissues of the body with a sufficient supply of blood, and specifically by impaired ventricular function, either due to reduced pump function (systolic dysfunction) or reduced filling (diastolic dysfunction).

Heart failure may be defined broadly as a condition where the heart is not able to pump blood to the rest of the body at a normal rate; it is the inability of the heart to pump out all of the blood that returns to it, at a normal rate. When the heart cannot pump all the blood it receives, excess fluid may back up in the veins and into the lungs and other parts of the body, and this may sometimes result in fluid accumulating in various parts of the body such as the lungs. This is called congestion, and it is for this reason that heart failure has in the past been referred to as “congestive heart failure”. However, not all patients experience problems with excess fluid or congestion, or the congestion may be controlled with drugs, and hence the term “heart failure” is preferred and is now more commonly used. The lack of blood being supplied to the body, in addition to the possible build-up of fluids, causes the symptoms of heart failure, which may include weight gain (due to fluid accumulation), swelling, difficulty in sleeping, tiredness, shortness of breath, particularly on exertion, and reduced capacity to carry out the normal tasks and activities of life.

There are a number of different causes of heart failure, including both ischemic and non-ischemic, of which the most common in the Western world is coronary artery disease and subsequent myocardial infarction. Other common causes are cardiomyopathy (primary or secondary), hypertension, valvular diseases, congenital defects, diabetes, thyroid diseases, alcohol abuse, certain cancer therapies, infections and illegal drug use.

Approximately 70% of new cases of heart failure in the Western world are caused by coronary artery disease, which is usually due to atherosclerosis. Atherosclerosis results in narrowing of the vessels in the heart, leading to inadequate blood supply to the myocardium (muscle cells). Such heart disorders which involve a reduced supply of blood to the heart are sometimes given the general term “ischemic heart disease”. Ischemic heart disease (or ischemic cardiomyopathy) is the major cause of heart failure in the Western world.

A reduced blood supply to the heart can manifest itself as angina pectoris (pain in the chest), acute myocardial infarction (which is the result of acute coronary artery occlusion causing irreversible damage to the myocardium with subsequent necrosis and loss of myocardial tissue), or sudden death. Since myocardial tissue has a very limited ability to regenerate, myocardial tissue lost by myocardial infarction will be replaced by scar tissue; such an area cannot sustain cardiac muscle function. If the blood supply to the heart is reduced over periods of weeks to years, or if the myocardium has been substantially weakened by infarction and replaced with scar tissue, cardiac function will become weakened with reduced pumping ability leading to the clinical manifestation of chronic heart failure.

Heart failure is characterised by impaired ventricular function, commonly left ventricular function although the right ventricle or both ventricles may be affected, increased peripheral and pulmonary vascular resistance and reduced exercise tolerance and dyspnea. As noted above, circulatory congestion may result from the decrease in cardiac output or from the damming of blood in the veins behind the left or right heart.

The more common forms of heart failure often cannot be cured, but treatment may improve or alleviate symptoms.

Current treatment for heart failure is based partially on preventative measures such as controlling diet, for example reducing or excluding caffeine and sodium, weight loss and exercise. Surgical means are used in more serious cases, for example coronary bypass surgery which eases symptoms by increasing blood flow to the heart, coronary angioplasty or transplantation if the heart has lost significant pumping capacity.

In terms of drug therapies, this has changed in recent years from treating haemodynamic imbalance to treatment aimed at preventing or correcting maladaptive biological responses, specifically neurohormonal changes brought about as a result of the heart failure. One such maladaptive response now well-recognised is in the adrenergic system; chronic β-adrenoceptor mediated signalling is believed to be a harmful compensatory mechanism in the failing heart and provides the rationale for β-blocker therapy in heart failure. Other maladaptive (or adaptive) changes may occur in the renin-angiotensin-aldosterone system.

Thus, for example, angiotensin converting enzyme (ACE) inhibitors slow the progression of heart failure by inhibiting the formation of angiotensin and causing vasodilation. Angiotensin receptor blockers (ARBs) may also be used. The use of diuretics is also common, which relieve water retention in the body thus easing the workload on the heart. Digitalis preparations such as digoxin are also used to increase the force of the heart's contractions.

β-blockers are commonly used, alone or in combination with ACE inhibitors. The failing heart is adrenergically activated, in contrast to the normally functioning human ventricle when in a resting state (Bristow, 2000, Circulation, Vol 101, 558-569). The increase in cardiac adrenergic drive appears to be damaging to the failing heart and is thus termed a maladaptive response. This response appears to be associated with changes in the composition of the adrenoceptors during heart failure with up-regulation of α₁ adrenoceptors and the down-regulation of β adrenoceptors. In addition, mouse models overexpressing activated adrenoceptors show cardiomyopathy and systolic dysfunction. Chronic adrenergic signalling is therefore considered to be a harmful compensatory mechanism in the failing human heart. In the end stage failing heart, 50-60% of the total signal transducing potential is lost. Blockade of the remaining signalling capacity using β-blockers complements the heart's endogenous antiadrenergic strategy of desensitisation, which is considered to be an adaptive change (Bristow, 2000, Circulation, Vol 101, 558-569). By inhibiting the remaining signalling potential of β adrenergic receptors using β blockers, a relatively effective method of treating heart failure has been developed and used with some success.

Nonetheless, whilst β blockers have been used with relative success, there is a continuing need for further drugs and treatments for heart disease. β blocker treatment is not successful for all patients as some patients show contraindications to β blockade such as reactive airways disease, sinus node or conduction system disease with bradycardia. Furthermore the target doses require careful manipulation and management for the desired result to be achieved and some individuals may not respond to β-blockade (Bristow, 2000, Circulation, Vol 101, 558-569). Despite the range of different pharmacological treatments now available, patients with symptomatic heart failure still have a high mortality, and accordingly there is a continuing need for new and effective treatments both as an alternative and more particularly to supplement existing treatments.

As noted above, coronary artery disease is a major cause of heart failure and may in itself cause death and morbidity. In particular, acute coronary syndromes, which result from acute obstruction (blockage) in a coronary artery, cause significant clinical problems. Acute coronary syndromes usually occur when an acute thrombus (blood clot) forms in an atherosclerotic coronary artery. Rarely, these syndromes may be caused by an arterial embolism. The thrombus abruptly interferes with blood flow to parts of the myocardium and whilst spontaneous thrombolysis may occur, in almost all cases the obstruction lasts long enough to cause damage to the heart (e.g. necrosis or damage due to ischaemia (reduction in blood supply)).

Initial consequences vary with the size, location and duration of obstruction and range from transient ischaemia to infarction. The clinical consequences range from unstable angina to myocardial infarction (MI) and sudden cardiac death.

The ischaemic event may thus vary in severity and whilst areas of ischaemia in the heart tissue may be reversible, the ischaemia may cause damage to the heart. Some necrosis is believed to occur even with mild ischaemia. The ischaemia may result in myocardial dysfunction, where the ischaemic tissue has impaired contractility and exhibits electrical dysfunction (is incapable of normal electrical activity).

Myocardial infarction (heart attack) occurs when the obstruction (e.g. a clot) is sufficiently severe, and may be described as the myocardial necrosis/cell death resulting from abrupt reduction in coronary blood flow. Apoptosis also plays a role in the process of tissue damage subsequent to myocardial infarction. Due to the limited capacity of myocardial tissue to regenerate, myocardial infarction leads to permanent loss of tissue and dysfunction of remaining myocardial tissue. Infarcted tissue is permanently dysfunctional. On the basis of ECG, a distinction may be made between ST elevation MI (STEMI) or non-ST elevation MI (NSTEMI) and both these conditions, along with unstable angina, may be viewed as sub-types of acute coronary syndrome.

Unstable angina (acute coronary insufficiency, pre-infarction angina, intermediate syndrome) is defined as rest angina which is prolonged (usually greater than 20 minutes), new-onset angina of at least class III severity in the Canadian Cardiovascular Society (CCS) classification system of angina pectoris, or increasing angina (e.g. previously diagnosed that has become distinctly more frequent, more severe, longer in duration, or lower in threshold (e.g. increased by one or more CCS class or to at least CCS class III). Unstable angina is clinically unstable and often a prelude to MI or arrhythmias, or less commonly to sudden death.

Thus, the impaired blood flow to the heart which occurs in an acute coronary syndrome may result in damage to the heart, and specifically to the muscle tissue of the heart (myocardium).

If impaired blood flow to the heart lasts long enough, it triggers the ischaemic cascade; the heart cells (myocytes) become damaged and may die (chiefly through necrosis) and do not regenerate. Damage may occur as a result of the ischaemia per se or as a result of oxidant damage during the subsequent reperfusion following ischaemia (a recognised phenomenon termed reperfusion injury) due to increased release of oxygen free radicals. A scar tissue rich in collagen replaces the damaged or dead myocardial tissue. As a result, the patient's heart will be permanently damaged. This scar tissue also puts the patient at risk for potentially life threatening arrhythmias, and may result in the formation of a ventricular aneurysm that can rupture with catastrophic consequences. Left ventricular remodelling which may occur after myocardial infarction leads to left ventricular dysfunction and eventually overt heart failure.

Ischaemic or reperfusion damage to the heart may also occur for other reasons beyond coronary heart disease (or more specifically ACSs). Heart surgery (for a variety of heart conditions e.g. valvular defects or diseases) is becoming increasing prevalent. Such surgery, or other surgeries, may require that the patient is placed on a heart-lung machine. Heart tissue can become damaged during surgery or whilst the patient is on a heart-lung machine as a result of ischaemia and/or reperfusion damage. As noted above, reperfusion injury occurs when blood flow is restored after a period of ischaemia.

Damage to the heart may also occur for other reasons, and is not limited to ischaemia or related causes. For example, myocardial damage may occur upon chronic increase in cardiac workload as the result of increased left ventricular afterload e.g. due to hypertension or aortic stenosis.

Thus, a variety of heart diseases (which term is used broadly herein to include any disease, condition, or disorder) may result in, or from, or may be characterised by, damage to the heart, specifically damage to the myocardium, and such diseases represent a significant and growing drain on clinical resources world-wide. There is thus a continuing clinical need, not only therapeutically to treat such diseases, but also to prevent or limit their occurrence or development. Such prophylaxis to prevent or limit damage to the heart (more specifically myocardial damage) is of particular clinical and economic importance. The present invention addresses this need in a surprising and unpredicted way and is based upon the identification of a novel and heretofore unforeseen cardioprotective factor.

Thus, in work leading up to the present invention it has surprisingly been discovered that Connective Tissue Growth Factor (CTGF) functions as a cardioprotectant to protect the heart from damage which may, for example, result from ischaemia and ischaemia/reperfusion injury or from other causes, such as increased cardiac workload or cardiac stress. Initial work showed that CTGF may up-regulate the expression of genes known to be cardioprotective, and that a gene program appears to be activated which results in inhibition of cardiac growth. This suggested that CTGF might play a role in pre-conditioning the heart by gene re-programming, to protect the heart from the development of disease or damage. Importantly, subsequent work, which underlies the therapeutic proposals now made in the present invention, showed that not only are gene effects involved, but that CTCF may have a direct acute effect on the heart. This direct effect may involve a direct agonistic effect on plasma membrane receptors present on cardiac myocytes, and the direct, rapid activation of signalling pathways (i.e. not via a gene effect). Thus CTCF may be used to treat damage which has already occurred or is occurring, to mitigate or reduce the effects of that damage, to reduce the extent of damage, or to prevent the initial damage from leading to further damage or disease of the heart, or to delay or reduce the extent of the further damage or disease, or indeed to improve the functioning of the heart after the heart-damaging event.

CTGF has a cardioprotective effect on heart (specifically myocardial) tissue and in addition to its prophylactic effects in preventing or limiting damage to the heart may also exert beneficial therapeutic effects on damaged heart tissue. In particular, it has been shown that CTGF activates signal pathways known to be protective after an injury. Thus, broadly speaking, a beneficial new therapeutic and prophylactic effect may lie in the use of CTGF as a cardioprotective agent, to protect the heart against damage, specifically myocardial damage, which may occur for example as a result of injury or disease. CTGF thus represents a surprising new pharmacological modality for the treatment (i.e. therapeutic treatment) and prophylaxis of myocardial damage. On this basis, CTGF may for example be used to treat, prevent, delay or reduce the development of heart failure or underlying conditions which may cause or lead to heart failure (e.g. ischaemic heart disease, ventricular remodelling or cardiomyopathy), in the treatment of acute coronary syndromes, for example to prevent or reduce myocardial infarction (e.g. to reduce infarct size), to treat myocardial infarction, and to protect the heart from possible damage during surgery.

More particularly, since, as noted above, it has surprisingly been discovered that CTGF can confer direct cardioprotective effects on the heart, this leads to the proposal that CTGF can be used in acute settings in which a patient is experiencing a heart-damaging event, or even after a patient has undergone a heart-damaging event. Thus, CTGF can be administered during or after a heart-damaging event, particularly an acute heart-damaging event such as, for example, acute myocardial infarction. Such administration may be beneficial in treating or reducing the damage, and, as mentioned above, it may also prevent or reduce further damage to the heart or may prevent, reduce or delay the development or progression of heart disease which may occur as a result of the heart-damaging event (e.g heart failure, as discussed further below) or more generally improve or normalize heart function. CTGF may be used to treat acute damage to the heart. CTGF has cardioprotective effects on myocardial tissue, and thus acute damage to myocardial tissue caused by acute cardiac damage can be ameliorated by CTGF. It can be administered acutely to patients, or on a short term basis. Alternatively, it may be administered on a more long term basis, for example to treat the effects of the damage, or to prevent further damage or disease from occurring, or to reduce further damage or disease or improve heart function. CTGF can promote the recovery of myocytes following a heart-damaging event, and this promotion of recovery may decrease the amount of damage to the heart, and particularly myocardium, following a heart-damaging event. Acute cardiac damage may result from, for example, myocardial infarction or other acute coronary syndromes or ischemia/reperfusion injury. The direct cardioprotective effects of CTGF may result from the activation of signal pathways known to be protective after an injury, as mentioned above.

CTGF is known as a growth factor. In general, growth factors are locally active intracellular signalling polypeptides which stimulate target cells to proliferate, differentiate and organise in developing tissues. Growth factors bind to cognate receptors on the cell surface, activation of which leads to an intracellular signalling cascade, ultimately resulting in the exerted effect of the growth factor on the cell.

CTGF is a member of the CCN family of growth factors which includes, in addition to CTGF (CCN2), Cyr61/cef 10 (CCN1) and Nov (CCN3), leading to the acronym CCN. Although grouped together in the same family, the various CCN proteins are distinct proteins encoded by separate genes. In particular, CCN1 (Cyr61) whilst also termed CTGF-2, is a protein distinct from CTGF, with different biological effects. The CCN family of proteins are believed to be involved in multiple cellular events including extracellular matrix (ECM) formation, cell adhesion, proliferation, or in some cell types, apoptosis. However, more than 15 years after its discovery, the precise physiological role of CTGF is unknown. This paucity of knowledge may be due to a variety of reasons. First, the CCN growth factors are glycosylated, cysteine-rich proteins, i.e. proteins notoriously difficult to express and purify in fully processed and active states. Secondly, the plasma membrane receptor and intracellular pathways for CTGF are poorly characterized. Several reports provide evidence that the CCN growth factors bind integrin receptors. However, the data is not entirely consistent. Reports also indicate that CTGF may bind the Wnt co-receptor LDL receptor-related protein 6 (LRP5/6), or TRK-A receptor tyrosine kinases.

CTGF has also been reported to synergize with TGF-β at the TGF-β receptors TβRI and TβRII, although the mechanisms of this synergism are not clear. Conceivably, as a protein consisting of several domains with different putative functions, the diverse reported interactions of CTGF may have its basis in the modular structure of CTGF.

In spite of the above difficulties, some information is available regarding the nature of CTGF. CTGF is a 38 kDa cysteine-rich secreted protein and is one of six distinct members of the CCN family of genes. The CTGF gene contains a TGF-β response element in its promoter region and is often considered to be a downstream mediator of some of the effects of TGF-β. The discovery of CTGF is described in U.S. Pat. No. 5,408,040 and it is proposed as a chemotactic and mitogenic agent for cells. Potential uses to induce the formation of connective tissue, including bone, cartilage and skin, are detailed in WO96/38168.

Thus, CTGF is produced by fibroblasts and endothelial cells, in the former in response to TGF-β. The primary biological activity of CTGF reported is mitogenticity, i.e. the ability to stimulate target cells to proliferate. The result of this mitogenic activity in vivo is the growth of the particular targeted tissue. CTGF also possesses chemotactic activity, i.e. the ability to induce movement of cells as a result of interactions with particular molecules. Several cell types appear to be responsive to CTGF, including fibroblasts, endothelial cells, chondrocytes and, as demonstrated in the work leading to this invention, cardiac myocytes.

Tissue levels of CTGF are elevated in various fibrotic disorders where excessive ECM formation is observed, and on this basis it has been hypothesized that CTGF may play a causative role in the development of such disorders, although a direct causative role of CTGF in fibrosis remains to be demonstrated. Nonetheless, therapies have been proposed, to combat fibrotic disorders, by blocking (antagonising) CTGF action.

Interestingly, CTGF is highly expressed in the myocardium during embryogenesis, but is repressed in the postnatal heart. Myocardial CTGF expression is, however, rapidly induced in heart failure of diverse etiologies. Indeed, myocardial CTGF mRNA levels have been shown to be a robust marker of heart failure with predictive power similar to myocardial mRNA levels of the natriuretic peptides ANP and BNP, and thus it had been thought that CTGF may play a causative role in the development of heart failure.

Indeed, the current state of the art reflects the hypothesis that CTGF may cause pathological fibrosis, and research in the area of CTGF and fibrosis in terms of therapy has focussed on inhibiting the activity of CTGF in an attempt to reduce fibrosis. For example, WO 2006/074452 discloses the regulation, particularly down-regulation, of CTGF (CCN2) by CCN3 in therapy for renal fibrosis and WO 2005/094796 discloses the use of SGK1 to prevent up-regulation of CTGF expression in fibroproliferative disorders. He et al. (2005) J Biol Chem 280(16):15719-15726) discusses the reversal of the increase in CTGF in cardiac tissues of streptozotocin-induced diabetic rats.

However, evidence for the pathophysiological role of CTGF in heart failure remains elusive. In order further to investigate the possible pathophysiological role of CTGF, and in particular to see the effects of specific over-expression of CTGF in the heart, the present inventors constructed a transgenic mouse model with cardiac-restricted expression of CTGF under the control of a cardiac-specific promoter (the myosin heavy chain promoter) (Tg-CTGF mice). The results obtained were very surprising, and rather than increased cardiac fibrosis restricting cardiac function, as might have been expected, CTGF was found to elicit a number of novel and totally unexpected effects which indicated that it was, on the contrary to a pathological role, functioning in a cardioprotective manner. Thus, totally unexpectedly, rather than leading to the progression of heart failure, myocardial CTGF appeared to be mediating cardioprotective actions.

It was surprisingly found that although the Tg-CTGF mice displayed a subtle increase of extracellular matrix proteins in the heart, cardiac fibrosis was inconspicuous and did not appear to affect cardiac function. The Tg-CTGF mice had slightly smaller hearts than non-transgenic control mice, with smaller dimensions, but unaltered cardiac function. Thus, expression of CTGF in the post-natal heart inhibits cardiac growth, a finding also reflected in the smaller dimensions of cardiac myocytes.

Furthermore, in investigating the effects of CTGF on gene expression in myocardial tissue; it was-also surprisingly found that CTGF causes the induction of a number of myocardial genes known to be involved in the regulation of cardiac growth and in cardioprotection.

Specifically, the upregulated genes included the G protein-coupled receptor kinase GRK5, which is documented to catalyse phosphorylation and desensitisation of β adrenergic receptors in cardiac myocytes. This prompted further work which revealed that the responses of cardiac myocytes from Tg-CTGF mice to the β agonist isoproterenol were blunted compared to non-transgenic mice, and that CTGF by way of increased myocardial GRK5 among other CTGF-regulated proteins with cardioprotective properties, conferred protection against chronic isoprotenerol-induced cardiotoxicity. Thus, the chronic isoprotenerol administration caused cardiac hypertrophy (as evidenced by left ventricular dilation and impaired systolic function) in the control hearts, whereas this was not seen in the transgenic hearts, where left ventricular dimensions and systolic function were preserved. Such induced cardiotoxicity is reminiscent of the maladaptive damage seen in heart failure resulting from chronic β-adrenergic receptor stimulation, and can be seen as a model of cardiomyopathy or heart failure.

As described in more detail in Example 1 below, in other models of cardiac damage, preservation of heart structure and function was also seen in transgenic as compared to non-transgenic control hearts. Thus, after chronic pressure overload induced in vivo by aortic banding, again CTGF protected against deleterious effects on both cardiac structure (geometry) and function, which were seen in the control mice. The Tg-CTGF mice displayed remarkable resistance to dilated cardiomyopathy and heart failure compared to the non-transgenic mice. Isolated hearts from Tg-CTGF mice were also shown to be protected against ischaemia/reperfusion injury.

This effect of CTGF has also been confirmed in studies with isolated hearts (from non-transgenic mice) perfused with CTGF as discussed in Example 1. Similar cardioprotective effects of CTGF administered exogenously were also observed. In order to confirm a direct cardioprotective action of CTGF in the heart which did not require longer term gene expression effects as with the transgenic mice, mouse hearts were subjected to Langendorff-perfusion ex vivo in the absence or presence of recombinant CTGF. It was found that mouse hearts that received recombinant CTGF recovered faster during subsequent reperfusion, generated significantly higher left ventricular-developed pressure and acquired smaller infarct size than control hearts. Thus, as discussed above, CTGF also has direct cardioprotective effects on the heart.

The results reported herein demonstrate that CTGF is a cardioprotective factor that may halt or delay the onset of heart failure or reduce myocardial infarction following ischaemia/reperfusion injury to the heart. As described herein, the mechanisms that may confer cardioprotection against ischaemia/reperfusion injury may also protect against pressure overload-induced cardiac dysfunction and heart failure. Thus, the results and effects reported and discussed herein directly support the therapeutic and prophylactic proposals set out above.

Accordingly, in one aspect the present invention provides CTGF or a nucleic acid molecule comprising a nucleotide sequence encoding CTGF for use as a cardioprotective agent. In particular, CTGF or the encoding nucleic acid molecule may be used in the treatment of a subject who has incurred or is incurring damage to the heart, wherein said CTGF or nucleic acid molecule is for administration during or after the heart-damaging event.

Alternatively viewed, this aspect of the present invention provides use of CTGF or a nucleic acid molecule comprising a nucleotide sequence encoding CTGF in the manufacture of a cardioprotective agent (or alternatively put, a medicament for use as a cardioprotective agent, or in cardioprotection). Again, the CTGF or encoding nucleic acid molecule is particularly used where the subject has incurred or is incurring damage to the heart, wherein said CTGF or nucleic acid molecule is for administration during or after the heart-damaging event.

Also provided is a pharmaceutical composition comprising CTGF or a nucleic acid molecule comprising a nucleotide sequence encoding CTGF for use as a cardioprotective agent, particularly wherein the composition is for use in the treatment of a subject who has incurred or is incurring damage to the heart, wherein said CTGF or nucleic acid molecule is for administration during or after the heart-damaging event. Such a pharmaceutical composition may further comprise at least one pharmaceutically acceptable carrier, diluent or excipient.

Still further is provided the use of CTGF or a nucleic acid molecule comprising a nucleotide sequence encoding CTGF as a cardioprotective agent.

The CTGF or a nucleic acid molecule comprising a nucleotide sequence encoding CTGF may be used according to the present invention in any method of treatment or prophylaxis of a subject involving cardioprotection, or a cardioprotective effect as described herein. Accordingly, in a further aspect, the present invention also provides a method of cardioprotection (or of achieving cardioprotection), i.e. protecting the heart, of a subject which method comprises administering CTGF or a nucleic acid molecule comprising a nucleotide sequence encoding CTGF to said subject. Particularly, the method comprises administering an effective amount of said CTGF or nucleic acid molecule. Even more particularly, the method is for treating a subject who has incurred or is incurring damage to the heart, said method comprising administering CTGF or said nucleic acid molecule to said subject, wherein the CTGF or nucleic acid molecule is administered during or after the heart-damaging event. The methods of the invention can also be used for protecting the heart during or after surgery, said method comprising administering CTGF or a nucleic acid molecule comprising a nucleotide sequence encoding CTGF to a subject immediately before, during or after surgery.

The CTGF or encoding nucleic acid may be used in a method of treatment or prophylaxis of damage to the heart in a subject, particularly myocardial damage, and more particularly myocardial damage resulting from injury or disease, particularly cardiovascular disease, or disease or injury of the heart, particularly acute damage such as that caused by an acute coronary syndrome.

In all such aspects of the invention, the CTGF or encoding nucleic acid may be used for the treatment of a subject who has incurred (or undergone or experienced) or who is incurring (or undergoing or experiencing) an acute heart-damaging event (or acute heart damage).

In a different aspect the present invention provides a transgenic animal which exhibits cardiac-restricted expression of CTGF. Thus, such an animal has been modified to introduce (or to express) a transgene (i.e. a “foreign” or “introduced” (more specifically “exogenously introduced”) nucleic acid molecule) encoding CTGF under the control of a cardiac-specific promoter.

The term “cardioprotection” as used herein refers to an effect of protecting the heart from damage, particularly acute damage, or of treating or alleviating damage to the heart (or more particularly of treating or alleviating the effects of damage to the heart). Cardioprotection can occur in an acute setting, e.g. where a subject is undergoing or has undergone damage to the heart and wherein the CTGF is administered acutely or on a short term basis. Alternatively, or additionally, the CTGF or encoding nucleic acid may be administered on a more long term basis, over a longer period of time. A “cardioprotective agent” thus mediates or has a cardioprotective effect, including a protective effect with respect to acute damage to the heart, or after a subject has experienced, or while a subject is experiencing, a heart-damaging event, particularly an acute heart-damaging event. “Protection” encompasses both preventing and limiting or reducing damage. Thus, absolute prevention of damage is not required, and protection may be seen as any degree of reduction in damage incurred (for example as compared with a subject or individual who has not been treated with (e.g. administered) the cardioprotective agent), for example the size of an infarction may be reduced and thus a reduction in damage to the heart is achieved. Also, heart function may be improved or normalized.

In one aspect, cardioprotection may thus be seen as rendering the heart less susceptible to damage. The amount or degree of damage may be limited or reduced, or in some cases damage may be prevented altogether. The amount of damage caused by a heart-damaging event may be limited or reduced, particularly myocardial damage may be limited or reduced. Alternatively, a cardioprotective agent may exert a therapeutic effect on damaged heart tissue. Thus, cardioprotection includes also an effect in alleviating or mitigating the effects of damage on the heart, or of reducing or ameliorating the damage. The recovery of cardiac myocytes may be improved or promoted. Viewed from this aspect, the invention may be seen as providing for the salvage of cardiac myocytes.

“Damage” to the heart includes any effect on the heart which impedes it from working normally (or properly) i.e. which prevents or reduces cardiac function, or which causes the heart to function in a reduced or less effective way. Thus, damage may be any effect which results in cardiac dysfunction.

“Acute damage” to the heart, and particularly acute myocardial damage, or an “acute heart-damaging event”, particularly an acute myocardium-damaging event, can be thought of as damage, or a heart-damaging event, which has an abrupt or sudden onset, and/or which is of short duration. Acute damage or an acute event may be rapidly progressive, and may require urgent care. An acute heart-damaging event may come on quickly, and can be intense, but can be of a short duration. Reference to “acute damage” or to an “acute heart damaging event” includes an acute illness or disorder, e.g. an acute coronary syndrome, for example myocardial infarction. The invention is particularly concerned with the treatment of such acute illnesses or events.

In particular, the damage to the heart is myocardial damage, in other words damage to the myocardium or to cardiac myocytes. Damage to the heart may be seen as cellular damage, for example mitochondrial damage or damage to other sub-cellular organelles or structures, cell death, apoptosis, necrosis or infarction, or as hypertrophy of the heart or a change in cardiac structure, geometry, size or dimensions (e.g. remodelling of the heart). For example, pathomorphological diagnosis of acute myocardial damage is based on the presence of necrotic, inflammatory and sclerotic alterations of cardiomyocytes which reflect the outcome of the damage to cardiomyocytes. For example, during myocardial infarction cell death due to prolonged ischaemia occurs. Microscopically this can be categorised as coagulation necrosis in which ghost-like cell structures remain after hypoxic insult or contraction band necrosis with amorphous cells that cannot contract anymore. In acute MI in the first six hours after coronary artery occlusion, coagulation necrosis can be seen with no cellular infiltration.

Damage to the heart may include ischaemic damage, namely damage (particularly cellular damage) seen as result of ischaemia, or damage due to reperfusion after ischaemia (ischaemia/reperfusion injury), or damage resulting from increased cardiac workload or cardiac stress, particularly chronic cardiac stress or a chronic increase in cardiac workload, damage resulting from cardiotoxic substances (e.g. chemotherapeutic drugs such as doxorubicin or anthracyclines, or indeed any other drugs, whether therapeutic or recreational, chronic alcohol abuse or drug abuse, or heavy metals, including iron and copper), as well as damage resulting from infection, e.g. viral infection, leading to myocarditis (e.g. cytomegalovirus or coxsackie virus). Heart damage may thus result from drugs. In this regard, some therapeutic drugs, for example breast cancer drugs and chemotherapy agents, for example the breast cancer drug Herceptin (trastuzumab) or other HER2 targeting agents, and chemotherapy agents such as anthracyclines, for example doxorubicin, daunorubicin and idarubicin, can have damaging side-effects on the heart. Also, antipsychotic drugs, NSAIDs and drugs for type 2 diabetes, may cause damage to the heart. Non-therapeutic drugs, for example cocaine, amphetamines and anabolic steroids can also have damaging effects on the heart.

Ischaemic damage or ischaemia/reperfusion injury may occur in ischaemic heart disease, e.g. coronary artery disease, and particularly in acute coronary syndromes or during surgery, for example surgery on the heart or when a patient is on a heart-lung machine.

Damage resulting from cardiac stress or increased cardiac workload may occur in heart or cardiovascular disease or injury and may result from any condition (i.e. disease or disorder) which increases the pressure on the heart (pressure overload), for example hypertension or aortic stenosis. In this setting, the myocardial tissue remaining viable after myocardial infarction may also be subject to increased workload due to increased peripheral vascular resistance.

Damage to the heart may also result from any negative or maladaptive response of the heart, for example to injury or disease. Such a maladaptive response may be a change (e.g. an increase) in neurohormonal signalling. Thus, as discussed above, for example, in response to heart failure, the heart attempts to compensate by increasing signalling through the β adrenergic system. This increase in adrenergic drive causes further damage to the failing heart. Thus damage to the heart may result from maladaptive changes in the adrenergic system, the renin-angiotensin-aldosterone system or the serotoninergic system e.g. from increased β adrenergic signalling or increased signalling via the 5-HT4 receptor.

Cardioprotection may thus include increasing the tolerance (or resistance) of cardiac myocytes (or more generally myocardial tissue) to damaging effects, and more particularly to hypoxia, ischaemia, increase in cardiac workload or stress or any toxic substances or molecules to which the heart may become exposed, for example drugs (both therapeutic and non-therapeutic) or as a result of ischaemia (of any cause), ischaemia/reperfusion, or disease, or maladaptive response, for example increased signalling molecules, or drug treatments). The direct cardioprotective effects of CTGF may promote the recovery of myocytes during or following a heart-damaging event. For example, CTGF may cause or help or promote myocytes at the site of damage to recover, rather than undergoing apoptosis. In this respect CTGF may help to salvage myocytes following or during damage to the heart. For example, the infarction size following myocardial infarction may be reduced if CTGF promotes the recovery of myocytes at the edge of the infarction, rather than the myocytes undergoing apoptosis or being involved in scar tissue formation. The border zone of cells surrounding the infarction may be pushed to survive by CTGF thereby reducing the ultimate infarct size and reducing the amount of damage to the heart. Even a small reduction in infarct size may be beneficial to patients.

The term “cardioprotection” as used herein does not include any angiogenic effects, namely an increase in vascularisation or growth of blood vessels.

Thus, according to the present invention CTGF or a nucleic acid molecule comprising a nucleotide sequence encoding CTGF may be used in the treatment or prophylaxis of damage to the heart (or more specifically the myocardium), particularly damage due to injury or disease, and particularly acute damage to the heart. The CTGF or encoding nucleic acid may thus be used in the treatment or prophylaxis of a heart disease which involves damage to the heart (i.e. which results in (causes), or from, or which is in any way associated with (e.g. is characterised by) damage to the heart). The CTGF or encoding nucleic acid may be used to treat damaged heart (or myocardial) tissue, or to protect the heart (or myocardium) from damage, particularly in acute settings wherein the subject has incurred or is incurring damage to the heart.

As used herein ‘treatment’ refers to reducing, alleviating, ameliorating or eliminating the disease (which term includes any disease, condition or disorder), or, one or more symptoms thereof, which is being treated, relative to the disease or symptom prior to the treatment. Treatment may include an improvement or increase in cardiac function or performance, and in particular ventricular function or performance, more particularly left ventricular function or performance.

“Prophylaxis” as used herein refers to delaying, limiting, reducing or preventing the disease or the onset of the disease, or one or more symptoms thereof, for example relative to the disease or symptom prior to the prophylactic treatment. Prophylaxis thus explicitly includes both absolute prevention of occurrence or development of the disease, or symptom thereof, and any delay in the onset or development of the disease or symptom, or reduction or limitation on the development or progression of the disease or symptom.

The subject of the treatment or prophylaxis may be any human or non-human animal subject, but preferably will be a mammal, and most preferably a human subject.

The damage may be manifest as dysfunction of the heart, particularly ventricular dysfunction, including right and/or left ventricular dysfunction, but particularly left ventricular dysfunction.

The damage may also or alternatively be manifest as a change in the size, structure, geometry or dimensions of the heart, for example as hypertrophy of the heart or as remodelling of the heart, particularly ventricular remodelling and especially left ventricular remodelling.

Such damage or changes to the heart may be seen as echocardiographic changes (specifically changes in echocardiographic variables or parameters) or as changes in haemodynamic variables or parameters. Such parameters or variables are used routinely in the art to assess heart function or damage, and are discussed further below.

CTGF or its encoding nucleic acid may thus be used in the treatment or prophylaxis of a range of heart diseases, including in particular heart failure or a disease or condition which predisposes or leads to heart failure (e.g. ischemic heart disease; cardiomyopathy; ventricular dysfunction (which may include both systolic dysfunction (reduced ventricular pump action, which may be defined also as reduced ventricular contractile function or reduced ventricular emptying) and/or diastolic dysfunction (reduced ventricular filling, or resistance to ventricular filling), particularly left ventricular dysfunction; and cardiac remodelling, particularly ventricular remodelling, and especially left ventricular remodelling) and acute coronary syndromes. (including unstable angina, and particularly myocardial infarction). CTGF or its encoding nucleic acid may also be used to protect the heart before, during or after surgery, or immediately before, during or after surgery, including during ex vivo transportation of an explanted heart.

The term “heart failure” as used herein defines a condition characterised by impaired cardiac function, specifically impaired ventricular function, either due to reduced pump action (systolic dysfunction) or reduced filling (diastolic dysfunction). Systolic dysfunction may be described as a condition of ventricular contractile dysfunction. Inadequate ventricular emptying is seen. Diastolic dysfunction may be described as resistance to ventricular filing. Heart failure may thus be seen as a ventricular condition or condition of ventricular failure.

The heart failure may be left-sided (left ventricular involvement or dysfunction) or right-sided (right ventricular involvement or dysfunction) or it may involve both sides of the heart (both right and left ventricles). Heart failure implies impaired function of the myocardium of the heart.

Particularly, chronic forms of heart failure (i.e. chronic heart failure) are concerned.

Thus, heart failure can be defined as a disorder which may result from any condition that reduces the ability of the heart to pump blood. Often the cause is decreased contractility of the myocardium resulting from diminished coronary blood flow (e.g. heart failure caused by coronary artery disease (CAD) or coronary ischemic disease), but failure to pump adequate quantities of blood can also be caused by damage to heart valves, external pressure around the heart, primary cardiac muscle diseases (e.g. idiopathic dilated cardiomyopathy) or any other abnormality which makes the heart a hypoeffective pump. Heart failure may be manifested as (or may result from) reduced cardiac output, and in particular a cardiac output which is inadequate to meet the demands of the body of a subject.

Thus included in the scope of the invention is heart failure caused by or resulting from ischemic heart disease (ischemic cardiomyopathy), particularly chronic ischemic heart disease, chronic non-ischemic cardiomyopathy including idiopathic dilated cardiomyopathy and cardiomyopathy due to hypertension.

Heart failure may be manifest in either of two ways: (1) by a decrease in cardiac output or (2) by a damming of blood in the veins behind the left or right heart. The heart can fail as a whole unit or either the left side or the right side can fail independently of the other. Either way this type of heart failure can lead to circulatory congestion and, as a result, has in the past been referred to as congestive heart failure.

Heart failure can be divided into two phases, acute (short term and unstable) and chronic heart failure (long term and relatively stable). The division between the two is difficult to define precisely, but generally acute heart failure is the stage of failure which occurs immediately after heart damage (i.e. has a rapid onset and short course) and is associated with instability in cardiac function and circulation, for example a sudden drop in cardiac output. Providing the acute phase is not so severe as to result in death, the sympathetic reflexes of the body are immediately activated and can compensate for the sudden loss in cardiac function.

After the first few minutes of an acute heart attack, a prolonged secondary state begins. This is characterised by a retention of fluid by the kidneys and by the progressive recovery of the heart over a period of several weeks to months up until the point at which the cardiac condition stabilises. This phase of stability is known as chronic heart failure. Although the heart has compensated and stabilised it is still weak and may become progressively weaker. Generally, a subject exhibiting symptoms of heart failure for greater than 3 months, or more preferably greater than 6 months, can be regarded as having chronic heart failure, providing that no further symptoms of acute heart failure such as angina or evidence of myocardial infarction have occurred during this 3 month or 6 month period.

This means therefore that although symptoms vary largely between patients, patients with chronic heart failure characteristically have a reduced cardiac function, and in particular reduced ventricular function. The most common manifestation of reduced cardiac performance is systolic dysfunction. For example, such patients display a reduced ventricular ejection fraction, particularly a reduced left ventricular ejection fraction (LVEF), when compared to a “normal” person who has not suffered from heart failure. In normal persons left ventricular ejection fraction is usually above 60%, while an ejection fraction less than 35%, more particularly less than 40% is characterized as systolic dysfunction. Thus, an LVEF of less than 35% or less than 40% is characteristic of reduced heart function in patients with heart failure, particularly chronic heart failure. Less common than systolic dysfunction is diastolic dysfunction, in which the ejection fraction is relatively preserved (left ventricular EF>40%) or normal, but where left ventricular filling is reduced.

Other characteristics of reduced cardiac function include a reduced right ventricular ejection fraction, reduced exercise capacity and impaired haemodynamic variables such as a decreased cardiac output, increased pulmonary arterial pressure and increased heart rate and low blood pressure, which are often observed in patients with chronic heart failure.

The New York Heart Association (NYHA) classification system divides heart disease into four classes, depending on the severity of disease. NYHA class I: Patient with cardiac disease but without resulting limitations of physical activity; Class II: Patient with cardiac disease resulting in slight limitation of physical activity. Class III: Patient with cardiac disease resulting in marked limitation of physical performance. They are comfortable at rest. Class IV: Patient with cardiac disease resulting in inability to carry on any physical activity without discomfort. Symptoms may be present at rest.

The invention may be used for the treatment or prophylaxis of all classes of heart failure, but particularly classes II-IV or III-IV. Thus the subject may be in any one or more of classes I to IV, but particularly will be in classes II-IV or III-IV.

Thus, the present invention may be used for the treatment or prophylaxis of any kind of heart failure, irrespective of cause or etiology. The resistance of the heart to heart failure, or to a cause of heart failure, may be increased. By way of representative example, post-infarction heart failure or heart failure induced by a constantly increased afterload, e.g. hypertensive heart failure, may be mentioned. The present invention may thus be used to treat established or symptomatic or overt heart failure, particularly chronic heart failure, but including also acute heart failure or heart failure which is evolving or developing, including incipient heart failure or heart failure which is asymptomatic. It may also be used to protect the heart from heart failure, for example to prevent or delay the onset of heart failure or to prevent, limit or reduce the development of heart failure, for example to reduce or limit the extent or degree to which the heart failure develops or to reduce the susceptibility of the heart to heart failure. Thus, for example the development of terminal heart failure may be delayed. Heart failure subjects the heart to chronic stress and the cardioprotective effect of CTGF may be used to increase the resistance of cardiac myocytes to the such stress. In one embodiment the invention is used for the treatment of acute heart failure.

As noted above, the invention may also be used to treat or prevent the underlying causes of heart failure, which include ischaemic and non-ischaemic heart diseases as listed above. Such causes include any condition which results in or causes a chronic or persistent increase in cardiac workload or cardiac stress. In this way heart failure can be prevented from occurring or developing.

Thus in one aspect the present invention may be used in the treatment or prophylaxis of cardiomyopathy, including ischaemic or non-ischaemic cardiomyopathy, for example dilated cardiomyopathy and more particularly idiopathic dilated cardiomyopathy (ICDM), and cardiomyopathy due to hypertension.

Thus, according to the present invention the heart can be protected from heart failure in cardiomyopathy (e.g. dilated cardiomyopathy).

The development or progression of heart failure may be associated with ventricular remodelling, particularly left ventricular remodeling, which manifests as gradual increases in left ventricular end-diastolic and end-systolic volumes, wall thinning, and a change in chamber geometry to a more spherical, less elongated shape. This process is usually associated with a continuous decline in ejection fraction. In one embodiment of the invention remodelling, and particularly ventricular, preferably left ventricular, remodelling may be reduced or prevented.

Remodelling may lead to ventricular dysfunction, particularly left ventricular dysfunction. In another embodiment of the invention, ventricular dysfunction is treated or prevented. By way of example, the invention may be used to protect the heart from dysfunction following damage, e.g. following chronic cardiac stress or increased cardiac workload (e.g. from increased pressure or pressure overload, e.g. hypertension or aortic constriction (stenosis)).

CTGF or its encoding nucleic acid may also be used according to the present invention in the treatment or prophylaxis of ischaemic heart disease, including chronic ischaemic heart disease and acute ischaemic heart disease, and particularly in the treatment of coronary artery disease, which may protect the heart from damage from the coronary artery disease. Thus, the present invention may prevent or reduce the development of heart failure in coronary artery disease or after a coronary event, or more generally protect the heart during a coronary event, or from damage resulting from a coronary event.

‘Ischemic heart disease’ refers generally to a condition of the heart characterized by reduced blood supply to the heart muscle (myocardium), usually due to coronary artery disease (atherosclerosis of the coronary arteries). As discussed above, damage to the heart may occur in ischaemic heart disease (or as a result of coronary artery disease).

Ischaemia may itself cause damage to the heart due to the reduced supply of blood or oxygen. In an important aspect the damage which is treated according to the invention, or alternatively viewed, the heart-damaging event, is ischaemia, and as discussed herein the ischaemia may result from any cause, for example, coronary artery disease, e.g artherosclerosis, thrombosis or embolism. However, as mentioned above, associated with ischaemia there may also be reperfusion injury, which occurs when blood flow to the heart is increased or re-established. Thus reperfusion may cause increased damage over the ischaemia itself. Reperfusion injury refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function. Reintroduction of oxygen following ischaemia may cause a greater production of damaging free radicals, resulting in reperfusion injury. With reperfusion injury, necrosis can be greatly accelerated. The damage of reperfusion injury is due in part to the induction of the inflammatory response to damaged tissues. White blood cells carried to the area by the newly returning blood release a host of inflammatory factors, for example interleukins, in addition to the free radicals that are induced in response to tissue damage. The restored blood flow reintroduces oxygen within cells which damages cellular proteins, DNA, and the plasma membrane. Damage to the cellular membrane may in turn cause the release of further free radicals. Such reactive species may also act indirectly in redox signaling to induce apoptosis. Leukocytes may also congregate in small capillaries, causing an obstruction which may lead to additional ischaemia. Infarction may result from ischaemia.

According to the present invention CTGF may prevent or reduce damage caused by reperfusion following ischaemia. Thus, CTGF or its encoding nucleic acid may be used to treat ischaemia in the heart (or a subject who has undergone a cardiac ischaemic event), to prevent, reduce or ameliorate damage resulting from the ischaemia, and also to prevent, limit or reduce damage occurring as a result of reperfusion. In other words, the heart may be protected from damage arising from ischaemia or ischaemia/reperfusion. The tolerance or resistance of the heart (myocardium) to ischaemic damage or ischaemia/reperfusion injury may be increased. Thus, prior to or during reperfusion, CTGF may be used, pre-emptively, to protect the heart against reperfusion injury. In this situation it can be seen that the CTGF may be used after the ischaemic event but prior to during reperfusion.

Thus, in one preferred aspect, CTGF may be used to precondition the heart so as to minimize reperfusion injury following ischemia and subsequent restoration of blood flow. Advantageously, in such a situation the CTGF may be used so as to take advantage of the direct effects of CTGF on the heart, i.e. the direct effects which may occur shortly after the administration of CTGF following an ischaemic event (or any acute event). Thus, CTGF may be administered immediately or shortly after the ischaemia or ischaemic event. This may have an immediate or rapid benefit in protecting the heart. The administration may be acute, or short term. However, as discussed in more detail below, prolonged administration of CTGF, or administration over a longer period of time, beyond acute or short term administration, may have benefits in improving heart function.

Such a situation may occur for example when a surgical or therapeutic intervention is made to restore (e.g. to increase or re-establish) coronary blood flow. Such an intervention may be, for example, a surgical procedure to re-open coronary blood vessels or otherwise to restore coronary blood flow, for example in percutaneous coronary intervention (PCI) or coronary artery bypass surgery, or a pharmacologic procedure through administration of thrombolytic drugs. In such settings, CTGF may be used immediately after re-opening of thrombotic vessels to alleviate reperfusion injury.

The invention may thus be used in the treatment or prophylaxis of acute coronary syndromes. As noted above, an ‘acute coronary syndrome’ is a condition arising from a blockage or obstruction (usually a blood clot) in a coronary artery. An acute coronary syndrome may present as an as yet undiagnosed condition of the heart manifested as a set of signs and symptoms, usually a combination of chest pain and other features, interpreted as being the result of abruptly decreased blood flow to the heart (cardiac ischemia). As noted above, the most common cause is a disruption of atherosclerotic plaque in a coronary artery. Acute coronary syndromes include unstable angina (UA, not associated with heart muscle damage), and myocardial infarction (MI) in which heart muscle is damaged, specifically ST segment elevation myocardial infarction (STEMI) and non-ST segment elevation myocardial infarction (NSTEMI).

In one important aspect, the invention thus provides a method of prophylaxis of damage to the heart which may result from or be caused by an acute coronary syndrome. CTGF or its encoding nucleic acid may thus be used to protect the heart from damage which may occur during or as a result of an acute coronary syndrome, e.g. MI. CTGF may also be used to treat an acute coronary syndrome, e.g. unstable angina or MI, for example an evolving MI.

In a preferred embodiment, the CTGF or its encoding nucleic acid may be administered in an acute coronary syndrome to protect the heart against MI. This may include preventing the occurrence of MI, or reducing the susceptibility of the heart to MI, or reducing infarct size. The present invention may thus be used to reduce infarct size in an acute coronary syndrome, and particularly in MI (e.g. during or after MI).

As mentioned above, CTGF or its encoding nucleic acid may also be used according to the present invention to protect the heart during surgery or recovery from surgery. CTGF may be used to precondition the heart, to prevent or reduce damage, and this may include damage which may occur during or after surgery. The direct effects of CTGF can precondition the heart if CTGF is administered immediately or shortly before the surgery, or during the surgery, for example when the patient is undergoing anaesthesia. Such damage may be ischaemic damage or ischaemia/reperfusion injury. As described above, CTGF or its encoding nucleic acid may be used in (or with) PCI, to prevent damage which may occur from reperfusion. Damage may however occur in other surgical procedures, for example in cardiac surgery (e.g. open-heart surgery) or any surgery in which the subject is placed on a heart-lung machine. Accordingly the term “surgery” is used broadly herein to include any surgery and includes the removal and ex vivo transportation of a heart, for example for transplantation purposes. The CTGF or its encoding nucleic acid may be administered prior to or during the surgery, at any appropriate or desired time during the surgical procedure. The CTGF may also be administered after the surgery. The direct effects of CTGF enable beneficial cardioprotective effects to be achieved when CTGF is administered shortly or immediately before the surgery, i.e. at a time when effects of CTGF in up-regulating or otherwise affecting gene expression could not be expected to occur, or to occur in sufficient time to exert a cardioprotective effect, for example during the surgery.

Cardioprotection may be assessed or determined by determining (e.g. measuring or assessing in any way) one or more parameters of cardiac function or cardiac damage. Such parameters include various echocardiographic or haemodynamic parameters or variables. Such parameters or variables may be determined and compared prior to and after administration of the cardioprotective agent to determine whether there has been a, or the extent of the, cardioprotective effect.

Thus, a variety of cardiac parameters may be perturbed when the heart, or more specifically the myocardium, is damaged or when cardiac function is reduced. Such parameters include, for example, ventricular pressure and volume. These are often referred to as pressure-volume relationships. Right and/or left ventricular parameters may be assessed, but particularly left ventricular parameters. Examples of these parameters include left ventricular systolic pressure (LVSP) and end-diastolic pressure (LVEDP); left ventricular developed pressure (LVDP), left ventricular end-systolic and end-diastolic volume, left ventricular (dP/dt)ₘₐₓ, the left ventricular end-systolic pressure-volume relation and elastance. In vivo cardiac function can be determined by simultaneous LV pressure-volume recording as described in Georgakopoulos et al. 1998 Am J Physiol 274(Pt 2):H1416-1422.

Other parameters indicating reduced cardiac function include a reduced left or right ventricular ejection fraction, reduced exercise capacity and impaired haemodynamic variables such as a decreased cardiac output, increased pulmonary arterial pressure and increased heart rate and low blood pressure. In a clinical setting non-invasive analyses of cardiac function are usually preferred, unless left ventricular catheterization is performed for specific reasons. Non-invasive investigations of cardiac function usually include echocardiography and or functional magnetic resonance imaging (MRI) of the heart. Echocardiographic and MRI parameters which may be determined include left ventricular diameter (in systole or diastole e.g. LVDs or LVDd), ejection fraction, fractional shortening, and cardiac output. Echocardiography may also allow Doppler analysis of mitral flow deceleration as well as tissue Doppler analysis of myocardial contractility.

Cardioprotection may be indicated by an increase of or in cardiac parameters which indicate an increase in cardiac performance or function, and which are usually decreased when the heart is damaged or dysfunctional e.g. in heart disease (such as heart failure or ACS). Conversely, cardioprotection may be indicated by a decrease or reduction of or in parameters which indicate damage to the heart and which are usually increased when the heart is damaged or dysfunctional. The increase or decrease may be qualitative or quantitative. The increase or decrease (when compared to a control subject to whom no CTGF or encoding nucleic acid has been administered) may, for example, be at least 10, 20, 30, 40, 50, 60, 70, 80 or 90%.

Parameters that are usually decreased when the heart is damaged or dysfunctional include parameters of ventricular function, particularly left ventricular function e.g. ejection fraction, particularly LVEF, and parameters of contractile function.

Parameters that are usually increased when the heart is damaged or dysfunctional include the level of cardiac markers present in the circulation. Cardiac markers or cardiac enzymes are proteins from cardiac tissue found in blood. These molecules are released from the heart into the bloodstream following damage to the heart and the blood levels of these proteins may increase over time, for example following myocardial infarction. Such markers include troponin sub-units I or T, and the hypertrophic markers ANP and/or BNP (which includes any form thereof e.g. proANP, proBNP or any fragment thereof) and α-skeletal actin. Thus, cardiac and/or hypertrophic markers can be assayed to determine cardioprotection relative to untreated subjects who do not receive CTGF.

Cardioprotection may accordingly result in a relative decrease in parameters that are elevated in an untreated patient relative to a treated patient, and/or a relative increase in parameters that are decreased in an untreated patient compared to a treated patient.

The cardioprotective effect of the substances indicated herein may also be analyzed by their ability to reduce myocardial infarct size. Infarct size may be determined by tissue Doppler echocardiography or contrast-enhanced (gadolinium contrast) MRI of the heart. The latter is now routine analysis of virtually all patients suffering from myocardial infarction.

As noted above, it is accordingly not to be expected that “treatment” in accordance with the present invention will result in a complete cure of the heart disease treated (e.g. in the case of heart failure) or the heart damage, and treatment in accordance with the present invention includes an improvement or alleviation of any one or more of the symptoms associated with the disease e.g. heart failure, or damage, and also an improved quality of life for the subject and, ultimately a prolonged lifetime and improved survival. Treatment in accordance with the present invention also includes an improvement or increase of the functionality of the heart or, in other words an improvement or increase in cardiac function or performance (especially as noted above ventricular and in particular left ventricular performance). In particular, treatment in accordance with the present invention may result in an improvement or increase in any one or more of the symptoms and parameters associated with heart damage or function, or the heart disease in question, and in particular symptoms and parameters relating to ventricular and particularly left ventricular function.

In particular, administration of CTGF or encoding nucleic acid in accordance with the present invention after or during a heart-damaging event (e.g. ischaemia or more particularly an acute coronary syndrome, or any acute heart-damaging event) may result in improvement, or normalization, of cardiac (heart) function. For example an improvement may be seen when a treated subject is compared with a subject who has undergone the heart-damaging event but who has not been treated with CTGF according to the present invention. Such an improvement may be seen in any of the parameters of heart (cardiac) function discussed herein, e.g. parameters relating to ventricular function, including particularly ejection fraction, e.g. left ventricular ejection fraction. The improvement may be seen with administration of CTGF during or shortly or immediately after the heart-damaging event, or the CTGF may be administered for an interval or period of time after the heart-damaging event. For example the CTGF may be administered on an acute, or on a short term basis, e.g. for a period of up to 6, 5, 4, 3, 2 or 1 month(s), or up to 1, 2, 3, 4, 5 or 6 weeks after the heart-damaging event (more particularly, an acute heart-damaging event). It may be possible to see an improvement in cardiac performance after administration of CTGF has ceased. In other words, if CTGF is administered for a period of time after the heart-damaging event (for example starting during or immediately or shortly after the event, and continuing for a time period after the event, e.g. for a period of weeks or months e.g for up to 2, 3, 4, 5 or 6 months, for example for one, two or three months), the improvement in cardiac function may be seen, or may be maintained after the administration has ceased, or at an extended time point after the event, e.g. 8, 9, 10, 11, 12 or more months after the event. It may not be necessary to administer the CTGF continuously for the improvement to be seen or maintained. However, the invention also covers the long-term, or continuous, administration of CTGF after the heart-damaging event, for example for longer than 6, 9, 12, 18 or 24 months. Thus, the invention does not preclude administration which is maintained or continued (i.e chronic administration).

As noted above, an important indicator of improved cardiac function is an increase in ventricular ejection fraction, and in particular left ventricular ejection fraction (LVEF). This can be assessed by standard methods well known and documented in the art, for example by echocardiography, ECG synchronized gated radionuclide ventriculography (MUGA scan), angiography or magnetic resonance (MR) imaging, and is normally carried out when the subject is at rest. RVEF may also be increased.

Other parameters may also be improved, and for which subjects may also be assessed, e.g. both before and after CTGF treatment include overall clinical status, for example clinical performance as evaluated by NYHA functional class. In other words the NYHA functional class of a patient may be assessed before and/or after CTGF treatment. Such a clinical evaluation may normally be carried out by a trained cardiologist.

Other parameters include exercise capacity, for example as measured by peak oxygen uptake and peak work load. Methods for measuring exercise capacity are well known and documented in the art. For example exercise testing can be carried out using an electrically braked bicycle ergometer. An exemplary protocol might consist of a starting work rate of 20 W increasing by 20 W every second minute until exhaustion (defined as an inability to keep the pedalling rate steady at 60 rpm). Oxygen uptake (VO₂) can be measured using for example the EOS/SPRINT system. Peak VO₂, is taken as the highest VO₂ observed.

As noted above, haemodynamic and echocardiographic parameters may also be assessed to indicate improved cardiac function. For example improved cardiac function may be indicated by a decrease in pulmonary capillary wedged pressure and/or in pulmonary artery pressure and/or an increase in peak heart rate, peak systolic blood pressure and mitral velocity deceleration time. Echocardiographic variables may conveniently be measured by echocardiography carried out by a trained cardiologist and haemodynamic variables can conveniently be assessed by right-sided heart catheterization according to standard techniques.

The “improvement” or “increase” (or where appropriate “decrease” (or reduction)) in a symptom or parameter includes any measurable improvement or increase or decrease (or reduction) when the parameter in question is compared with the equivalent parameter in a non-treated individual or when the parameter in question is compared with the equivalent parameter in the same individual taken at an earlier time point (e.g. comparison with a “base line” level). Preferably the improvement or increase will be statistically significant. Especially preferably the improvement or increase or decrease in the symptom and/or parameter will be associated with the improved health of the subject concerned and more preferably a prolonged survival.

Methods of determining the statistical significance of differences in parameters are well known and documented in the art. For example herein a parameter is generally regarded as significant if a statistical comparison using a two-tailed significance test such as a Student t-test or Mann-Whitney U Rank-Sum test shows a probability value of <0.05.

In one aspect the patient or subject may be identified as in need of cardioprotection (e.g. as suffering from heart damage, or from a heart disease in question, or as being at risk of developing, or susceptible to, heart damage, or from a heart disease in question), before the CTGF or encoding nucleic acid is administered.

Such identification can be on the basis of symptoms and/or parameters which are indicative of cardiac damage or dysfunction as discussed above.

As discussed above, cardiac performance may be increased following the administration of the CTGF or its encoding nucleic acid. Accordingly, the various aspects of the present invention as presented and discussed above may further include assessing the subject being treated for an improvement in cardiac performance, or in the heart damage, or in the heart disease in question following administration of the CTGF or encoding nucleic acid. As discussed above, this assessment may be an assessment for an improvement in cardiac performance or function, particularly ventricular and especially left ventricular, performance or function or for an improvement in any symptom or parameter of heart damage or disease, as discussed above.

Where the subject is at risk of developing a heart disease, e.g. heart failure, or is susceptible to the heart disease e.g. heart failure, the subject may be assessed for one or more factors which are risk factors for heart disease in question. For example, for heart failure these may be ischaemic disease or conditions, e.g. coronary artery disease, cardiomyopathy, hypertension, valvular disease, congenital heart defects or any other predisposing condition or factor known in the art or described or mentioned above.

Following administration of the CTGF or encoding nucleic acid, the subject may be assessed for the development of the heart disease, e.g. heart failure or for one or more risk factors for the heart disease, e.g. heart failure.

The mechanism by which the CTGF exerts a cardioprotective effect has not yet been fully elucidated, but in any event, it is not critical to the proposed new therapeutic and prophylactic approach. However, without wishing to be bound by theory, the data obtained by the inventors suggests that CTGF may confer cardioprotection by pre-emptive preconditioning of the heart due to activation of Akt/GSK3-β signalling pathways and reprogramming of gene expression. Thus, as discussed above, there may be a direct effect on the heart, which may take place and may be observed within a short time period after CTGF administration e.g. after about, or after at least, 40, 45, 60, 90 or 120 minutes, and there may also be a more long-term effect on gene expression. The direct effect on the heart will be observed before 6, 9, 12 or 24 hours or more after CTCF administration, before an effect on gene expression would be expected to be seen. It is believed that CTGF may activate Akt/PKB phosphokinase cascades with subsequent phosphorylation and inhibition of GSK3-β. Expression of genes known to be cardioprotective appears to be up-regulated, for example, genes encoding free radical scavengers. Furthermore, a gene program appears to be activated, which results in inhibition of cardiac growth. It is thought that the effects on gene expression may take 40 to 48 hours to manifest. Thus effects on gene expression may be distinguished from the direct effects on the heart.

With regard to how the direct cardioprotective effects of CTGF are achieved, again this is not critical to the surprising new protective effect of the present invention, which may be seen during or after damage to the heart has occurred. Without wishing to be bound by theory, however, it is proposed that CTGF may have direct effects on the phosphorylation state of the signalling molecules involved in cardioprotection. Ion channels and kinases involved in the signalling involved in cardioprotection may be directly affected by CTGF. It is hypothesized that CTGF may act as an agonist at receptors for proteins involved in cardioprotection, or more generally as an agonist of such proteins, for example Akt and/or GSK-3β. CTGF is shown in Example 2 below to activate the Akt/GSK-3β pathway.

The term “CTGF” is broadly used herein to include all known forms of the CTGF polypeptide, as identified also by the term “CCN2”, and includes also functionally equivalent variants, derivatives and fragments thereof. Thus the term “CTGF” as used herein includes amino acid sequence variants of known CTGF polypeptides, and fragments of a CTGF polypeptide, or derivative thereof, as long as such fragments, variants or derivatives are active, or “functional”, i.e. retain at least one function or activity (e.g. biological activity) of a CTGF polypeptide. Such an activity may be any activity of CTGF, for example as may be determined in an in vitro assay, e.g. mitogenic or chemotactic activity or an activity in stimulating growth of fibroblasts. Such assays are known and described in the art (see for example Ahmed, M. S., Øie, E., Vinge, L. E., Yndestad, A., Andersen G. Ø., Andersson Y., Attramadal, T., and Attramadal, H. J, Mol Cell Cardiol 36: 393-404, 2004). Alternatively CTGF activity may be assessed or determined by assessing or determining a cardioprotective effect or activity of CTGF, as described herein, for example an effect or activity in altering expression of a gene as described herein or in modulating a signalling pathway, particularly activating a Smad2 or Akt/GSK-3β pathway, or in protecting an isolated heart against ischaemia/reperfusion injury by determining the effect on infarct size or recovery of contractility following re-perfusion, for example as detailed in Example 1 below. To assess CTGF activity, for example of a purified recombinant CTGF preparation or a variant or fragment thereof, any known assay for CTGF activity could be used, for example based on known biological effects of CTGF. For example, CTGF activity may be tested in concentration-effect analysis. The effect could be mitogenic activity as indicated above or phosphorylation of Akt (ser 473) or GSK-3β (Ser 9). Maximal activity (efficacy) and potency (concentration eliciting half-maximal effect) may then be compared between different CTGF preparations, fragments, variants or derivatives etc.

CTGF is a known protein and has been described in the literature, as discussed above. (Insofar as the referenced patent specifications refer to CTGF proteins and fragments or variants or derivatives thereof, they are incorporated herein by reference.)

Various fragments of CTGF have also been described, and reported in the literature to retain activity.

CTGF comprises 349 amino acid residues which are organised into a signal peptide and four structural modules that resemble an insulin-like growth factor-binding domain (module 1), a von Willebrand factor type C repeat (module 2), a thrombospondin type I repeat (module 3), and a C-terminal domain that contains a putative cysteine knot (module 4) (Bork P, FEBS Letters 327:125-130, 1993). Fragments representing or containing (comprising) such individual modules or domains can be obtained by routine methods known in the art, for example by recombinant expression. Module 4 may be referred to as module 3 in alternative terminology that is used in the art.

The full length CTGF polypeptide, excluding the signal peptide, may include amino acids 27-349. Module 1 may include amino acids 27-101 and module 2 may include amino acids 94-198. Module 3 may include amino acids 193-258 and module 4 may include amino acids 249-349 (Hoshijima et al. FEBS Letters 580 1376-1382 (2006)), or alternatively module 4 may include amino acids 247-349 (i.e. 102 amino acids corresponding to exon 4) (Ball et al. J Endocrinology 176:R1-R7(2003)). Each such module may represent a fragment which may be used according to the present invention. As a further alternative, a fragment corresponding to module 4 may be or may comprise a 98 amino acid peptide encoded by the last exon of the CTGF gene; this peptide has a molecular weight of 11.2 kDa. This 11.2 kDa form of CTGF is available from PeproTech, Rocky Hill, N.J., USA or Cell Sciences, Inc, Canton, Mass., USA (Sheng-Hua Wu et al. Growth Factors 26(4):192-200 (2008)).

Fragments of CTGF which retain activity are described in Gao and Brigstock, J. Biol. Chem. 279:8848-8855 (2004). Such fragments may include fragments comprising each of the four structural modules or domains, or combinations thereof. Thus, fragments of CTGF may include CTGF comprising modules 3 and 4, or only module 4 as described above.

A further fragment of CTGF may comprise residues 257-272 of the amino acid sequence of CTGF, which reside in module 4. This fragment of CTGF may comprise the amino acid sequence IRTPKISKPIKFELSG (SEQ ID NO:5).

Fragments of CTGF may also include a C-terminal domain and an N-terminal domain of CTGF (Grotendorst et al. FASEB J 19:729-738 (2005)). These individual domains of CTGF retain specific biological function when separated. The individual domains can be obtained by a variety of methods known in the art, for example, chymotrypsin or plasmin proteolysis of intact recombinant CTGF followed by separation of the pure individual domains by affinity chromatography using heparin Sepharose. If chymotrypsin digestion is used, a C-terminal domain with its N-terminal sequence beginning at position 181 (AYRLED—SEQ ID NO:6) relative to the initiation methionine can be generated. Alternatively, the C-terminal and N-terminal domains can be produced by expressing the domains individually by expressing only a limited region of the CTGF open reading frame by molecular biological methods known in the art.

The N-terminal contains two distinct structural motifs, the first of which is similar to one found in the IGF binding protein which is responsible for binding IGF. The second motif is related to the von Willebrand factor type C motif. Thus, an N-terminal domain fragment may comprise modules 1 and 2 as described above. The C-terminal domain contains a motif related to the thrombospondin-1 motif and a cysteine knot. Therefore, a C-terminal domain fragment may comprise modules 3 and 4 as described above.

WO 00/35939 describes fragments which have mitogenic activity, for example which comprise at least exon 4 or exon 5 of CTGF. Such fragments are included herein.

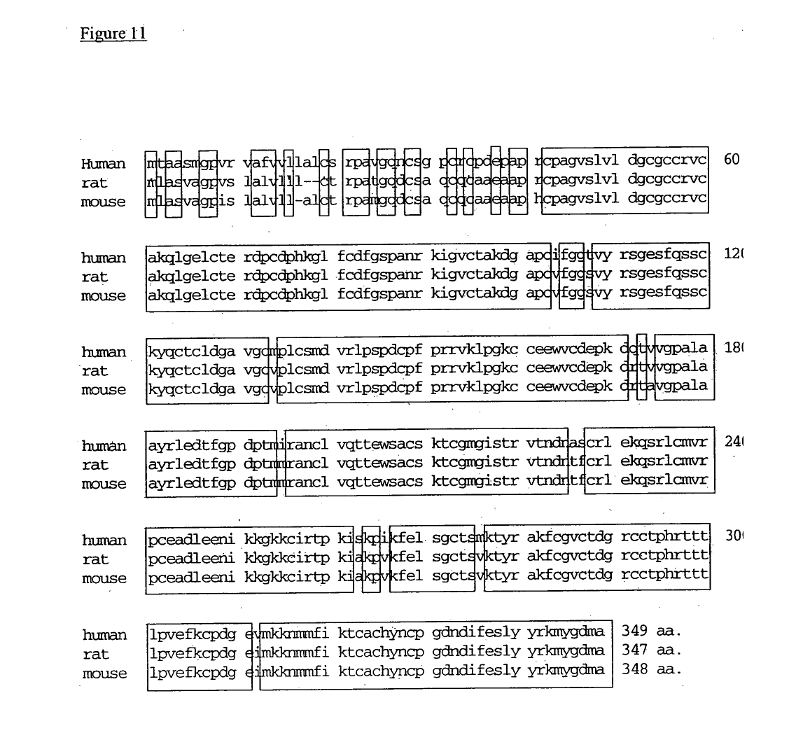

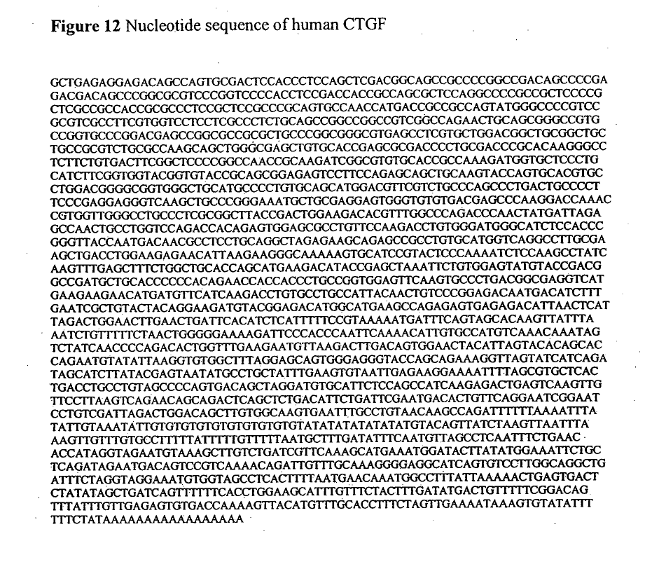

The amino acid sequences of CTGF from human and various other species have been elucidated and are publicly available, as are their encoding DNA sequences. FIG. 11 shows an alignment of the amino acid sequences of human CTGF (SEQ ID No. 1), rat CTGF (SEQ ID No. 2) and mouse CTGF (SEQ ID No. 3). The nucleotide sequence encoding human CTGF is shown in FIG. 12 (SEQ ID No. 4).

The CTGF may be a recombinant polypeptide, a synthetic polypeptide or may be isolated from a natural source.

The CTGF may be from any species (more particularly any vertebrate species), but preferably will be mammalian, and more preferably human.

In particular, the CTGF as used herein has an amino acid sequence as shown in any sequence of FIG. 11 (SEQ ID NO 1, 2, or 3), particularly SEQ ID NO. 1 or a functionally equivalent variant, derivative or fragment thereof.

Variants of CTGF may include, for example, different allelic variants as they appear in nature e.g. in other species or due to geographical variation etc. Functionally equivalent variants may also include polypeptides which incorporate one or more amino acid substitutions, or intrasequence or terminal deletions or additions to the above sequence.

Functionally equivalent derivatives may include chemical modifications of the amino acid sequence, including for example the inclusion of chemically substituted or modified amino acid residues.

A derivative may also be a molecule which is a peptidomimetic of a CTGF polypeptide. In other words, it may be a molecule which is functionally equivalent or similar to a polypeptide and which can adopt a 3-D structure which is similar to its polypeptide counterpart, but which is not composed solely of amino acids linked by peptide bonds. Thus, a peptidomimetic may be composed of sub-units which are not amino acids but which are structurally and functionally similar to an amino acid. The backbone moiety of the subunit may differ from a standard amino acid, e.g. it may comprise one or more nitrogen atoms instead of one or more carbon atoms. Furthermore, derivatives may include β-amino acids, which have their amino group bonded to the β carbon rather than the α carbon.

A preferred class of peptidomimetic is a peptoid, i.e. an N-substituted glycine. Peptoids are closely related to their peptide counterparts but differ chemically in that their side chains are appended to nitrogen atoms along the backbone of the molecule, rather than to the α-carbons as they are in amino acids.

All such variants and derivatives are included provided they retain an activity of CTGF, and particularly a cardioprotective activity. By way of example, a functionally equivalent variant, derivative or fragment may exhibit at least 10%, 20%, 30% or 40%, preferably at least 50% or 60% or 70% of the activity of a CTGF as shown in FIG. 11 or any one of SEQ ID NO. 1, 2, or 3, particularly SEQ ID. NO. 1.

It is known in the art to modify the sequences of proteins or peptides, whilst retaining activity and this may be achieved using techniques which are standard in the art e.g. random or site directed mutagenesis, cleavage and ligation of nucleic acids, chemical peptide synthesis etc.

Preferably, amino acid changes are of a minor nature, that is conservative amino acid substitutions that do not significantly affect the folding and/or activity of the protein; small deletions, typically of 1 to 30 amino acids; small amino- or carboxyl-terminal extensions; addition of a small linker peptide of up to about 20-25 residues; or a small extension that facilitates purification by changing net charge or another function, such as a poly-histidine tract, an antigenic epitope or a binding domain. Hence, N and/or C extensions to the protein or peptides are included in the definition. The lengths of each extended derivative may vary, for example, derivatives may be extended by up to 50, 30, 20, 10 or 5 amino acids. The use of fusion proteins comprising CTGF is further included according to the present invention, particularly a fusion protein comprising a polypeptide of SEQ ID NO. 1 or a functionally equivalent variant, derivative or fragment thereof.

Examples of conservative substitutions are within the group of basic amino acids (such as arginine, lysine and histidine), acidic amino acids (such as glutamine and asparagine), hydrophobic amino acids (such as leucine, isoleucine and valine), aromatic amino acids (such as phenylalanine, tryptophan and tyrosine) and small amino acids (such as glycine, alanine, threonine and methionine).

The CTGF preferably has at least 50%, 60%, 70%, 80% or 90% sequence identity or similarity to an amino acid sequence of SEQ ID NO. 1, 2 or 3 as shown in FIG. 11, particularly SEQ ID NO. 1, or a part thereof. More particularly, the CTGF has at least 95, 97, 98 or 99% identity or similarity to the sequence of SEQ ID NO. 1, 2, or 3, particularly SEQ ID NO. 1, or a part thereof.

Alternatively viewed, CTGF can be encoded by all or part of the nucleotide sequence shown in FIG. 12 (SEQ ID NO. 4) or a nucleotide sequence having at least 50%, 60%, 70%, 80% or 90% identity thereto. Preferably, the CTGF is encoded by a nucleotide sequence which has at least 95, 97, 98 or 99% identity to all or part of a nucleotide sequence as shown in FIG. 12 or SEQ ID NO. 4. The nucleotide sequence may be of genomic, cDNA, RNA or synthetic origin or any combination thereof.

The degree of identity between two nucleic acid and two amino acid sequences may be determined by means of computer programs known in the art such as GAP provided in the GCG program package (Needleman and Wunsch, 1970, Journal of Molecular Biology 48: 443-453). For the purposes of determining the degree of identity between two nucleic acid sequences for the present invention, GAP may be used with the following settings: GAP creation penalty of 5.0 and GAP extension penalty of 0.3. For the purposes of determining the degree of identity between 2 amino acid sequences, GAP can be used with the following settings: GAP creation penalty of 3.0 and GAP extension penalty of 0.1. Amino acid similarity may be measured using the Best Fit program of GCG Version 10 Software package from the University of Wisconsin. This program uses the local homology algorithm of Smith and Waterman with the default values: Gap creation penalty=8, Gap extension penalty=2, Average match=2.912, Average mismatch=2.03.

The CTGF may also be encoded by a nucleotide sequence that hybridises to a nucleotide acid sequence of FIG. 12 or SEQ ID NO. 4 under high stringency conditions defined herein as: prehybridisation and hybridisation at 42° C. in 5×SSPE, 0.3% SDS, 200 μg/ml sheared and denatured salmon sperm DNA and 50% formamide. The carrier material is washed three times for 30 minutes using 2×SSC, 0.2% SDS at least 70° C.

CTGF used in the present invention may be prepared synthetically by established techniques or by recombinant technology. Hence, CTGF may be produced recombinantly from its encoding nucleic acid which can also be produced synthetically e.g. in an automatic DNA synthesizer or may be isolated and cloned from genomic DNA. The nucleic acid can be inserted into a recombinant expression vector, e.g. a plasmid, where the nucleic acid encoding CTGF may be operably connected to a suitable promoter to allow expression in a particular cell. Techniques and materials for recombinant expression are well known, and any desirable or convenient vector may be used. The vector may for example be a plasmid, bacteriophage, or cosmid into which a nucleic acid (encoding the CTGF) may be inserted or cloned. Such vectors preferably contain one or more unique restriction sites and may be capable of autonomous replication in a defined host cell including a target cell or tissue or may be integratable with the genome of the defined host such that the cloned sequence is reproducible. The choice of the vector will depend on the compatibility of the vector with the host cell into which the vector is to be introduced. The vector may also include a selection marker and regulatory elements to control expression of the inserted nucleic acid. Examples of suitable promoters for transcribing the nucleic acid sequence in mammalian cells include the SV40 promoter, the MT-1 promoter, Rous Sarcoma virus promoter, cytomegalovirus promoter and a bovine papilloma virus promoter. Suitable promoters for expression in bacteria include for example the promoter obtained from the E. coli lac operon.

Hence, expression of CTGF may occur from its encoding nucleic acid e.g. from a vector containing the nucleic acid in a host cell. Such a cell may be prokaryotic or eukaryotic and may be mammalian, insect, bacterial or fungal.

CTGF produced recombinantly may be purified by any desirable or known means, for example by heparin affinity chromatography with a salt gradient for elution and subsequent ion exchange chromatography (S-Sepharose) or size exclusion chromatography e.g. using a Bio-Sil TSK-125 size exclusion column (Bio-Rad Laboratories) and eluted using an HPLC system (Ahmed, M. S., et al, J. Mol Cell Cardiol 36: 393-404, 2004).

CTGF may be administered according to the present invention as a polypeptide molecule, or it can generated “in situ” in the subject by administering a nucleic acid molecule comprising a nucleotide sequence encoding CTGF. Thus the present invention encompasses use of CTGF in methods of gene therapy. The nucleic acid molecule is administered to the subject, and is expressed in the subject, to produce CTGF in the subject.

The nucleic acid molecule may comprise a nucleotide sequence encoding any CTGF polypeptide (including a CTGF fragment) as discussed above. More particularly, the nucleic acid molecule may comprise a nucleotide sequence encoding all or part of an amino acid sequence as shown in any one of SEQ ID. NOS 1, 2 or 3, particularly SEQ ID NO. 1, or an amino acid sequence having at least 50% sequence identity thereto, or more as discussed above.

More specifically, the nucleotide sequence may be all or part of a nucleotide sequence as shown in SEQ ID NO. 4; or a sequence having at least 50% identity thereto (or more as discussed above) or a sequence which hybridises to the sequence of SEQ ID NO. 4 under conditions of high stringency (again, as discussed above).

The nucleic acid molecule may be administered in the form of, or contained in or on, a vector, and a number of vectors for use in gene therapy are known and described in the art.

The vector may comprise further elements for example expression control elements e.g. transcriptional and/or translational control or regulatory elements for expression of the nucleic acid molecules. Such control elements, e.g. promoters, ribosome binding sites, enhancers, terminators etc. are well known and widely described in the art.

The vector for example may be a virus or virus-derived vector, for example selected from a retrovirus, an adenovirus and an adeno-associated virus.

Other mechanisms and means by which nucleic acids may be administered for the purposes of gene therapy are known and described in the art and include cationic lipid-mediated gene delivery or copolymeric gene carriers. The naked DNA is in the form of a supercoiled plasmid DNA encoding the protein of interest under a strong eukaryotic promoter (e.g. cytomegalovirus promoter).

The nucleic acid molecule may be administered or delivered to the subject in a manner so as to achieve targeted gene expression, or expression in a desired target tissue, for example the heart. Thus, the nucleic acid molecule may be administered to the subject by or using a means which achieves targeted delivery of the nucleic acid to a target site in the body, for example using a targeted vector, or a vector which comprises target-specific expression control sequences (e.g. a target specific promoter, e.g. cardiac specific). Thus, the nucleic acid may be administered to the heart, or may be administered by a means which results in delivery to the heart, or specific expression in the heart.

It may also be desired to express the CTGF at (and hence deliver the nucleic acid to) a site from which CTGF may be released or secreted into the circulation. The expressed CTGF may thus be delivered to the heart via the circulation. Such a site may for example be muscle tissue, e.g. in the leg, or arm or elsewhere on the body. The nucleic acid may thus be administered to the desired site (e.g. muscle) and/or may expressed at that site using a tissue-specific (e.g. muscle-specific) promoter or other expression control element.

The use of gene therapy in this way (i.e. the use of the nucleic acid molecule according to the invention) may allow CTGF to be administered or delivered to the subject over a prolonged or extended period of time, which may depend upon the route of administration for example, for a few weeks, e.g. 1, 2, 3 or 4 weeks, for example in the case of naked plasmid DNA, or several months, for example in the case of a recombinant viral vector, e.g. a recombinant adeno-associated virus, e.g. 3, 4, 5, 6, 7, 8, 9, 10, 11, and up to 12 months.

Stem cells have in recent times been proposed as a possible therapy for various heart and other conditions. This leads to the proposal that a nucleic acid molecule encoding CTGF could be introduced into a stem cell or other cell (e.g. cardiomyocyte or a progenitor thereof) to enable that cell to express CTGF, and such a transformed or modified cell could be administered to the heart.

Accordingly, in another aspect the present invention provides a stem cell, or a cardiomyocyte or progenitor thereof (preferably a human cell, but in one embodiment not including a human embryonic stem cell) which has been modified by the introduction of a nucleic acid molecule comprising a nucleotide sequence encoding CTFG. Such a nucleic acid molecule may be any nucleic acid molecule as defined herein.

For use according to the present invention the CTGF or encoding nucleic acid may be formulated as a pharmaceutical composition. Such a composition may be formulated in any convenient manner according to techniques and procedures known in the pharmaceutical art, e.g. using one or more pharmaceutically acceptable carriers, diluents or excipients. “Pharmaceutically acceptable” as referred to herein refers to ingredients that are compatible with other ingredients of the compositions as well as physiologically acceptable to the recipient. The nature of the composition and carriers or excipient materials, dosages etc. may be selected in routine manner according to choice and the desired route of administration, purpose of treatment etc. Dosages may likewise be determined in a routine manner and may depend upon the nature of the molecule, purpose of treatment, age of patient, mode of administration etc.

CTGF or its encoding nucleic acid may be administered by any suitable method known in the medicinal arts, including oral, transmucosal, topical, or parenteral administration (e.g. intravenous, intramuscular, intraperitoneal or subcutaneous administration) or by inhalation. Preferably, the CTGF or nucleic acid is administered intravenously or via a means of direct delivery to the heart (e.g. intracoronary administration through a catheter positioned in a coronary artery; usually the left coronary artery) or by intramuscular, intraperitoneal or subcutaneous injection.

Administration of CTGF or nucleic acid may be in a single dose to be taken at regular intervals or may be administered as divided doses to be taken for example during the course of a day (e.g. 1 to 4 times a day). Alternatively, a sustained release formulation may be used which may be given at longer intervals (eg. once a day, or once every 2, 3, 4, or 7 days or more). The precise dosage of the active compound to be administered, the number of doses and the length of the course of treatment will depend on a number of factors, including age and size of the subject. However, preferably, a typical dose will result in tissue levels of CTGF of 10 to 100 nmol/L i.e. 10, 20, 30, 40, 50, 60, 70, 80, 90, or 100 nmol/L. The dose of CTGF to be administered can be from 10 to 100 nmol/kg body weight/min, i.e. 10, 20, 30, 40, 50, 60, 70, 80, or 90 nmol/kg/min for intravenous infusion in man (10 min infusion period). However, the dosage, intervals, and infusion time will depend on the pharmacokinetics of the administered CTGF in the circulation.

The compositions may comprise any known carrier, diluent or excipient. For example, formulations which are suitable for parenteral administration conveniently comprise sterile aqueous solutions and/or suspensions of pharmaceutically active ingredients preferably made isotonic with the blood of the recipient, generally using sodium chloride, glycerin, glucose, mannitol, sorbitol and the like.

When administered orally, the composition may be in the form of a tablet, capsule, powder, solution or elixir. When administered in tablet form, the pharmaceutical composition may additionally contain a solid carrier such as a gelatin or an adjuvant.

Generally speaking, for oral administration, the composition may need to be provided with a coating or in a form which provides protection from enteric degradation or digestion. The tablet, capsule or powder may contain from about 5 to 95% of the active ingredient. When administered in liquid form, a liquid carrier such as water, petroleum, oils of animal or plant origin such as peanut oil, mineral oil, soybean oil or sesame oil or synthetic oils may be added. The liquid form of the composition may also contain physiological saline solution, dextrose or other saccharide solution or glycols. When administered in liquid form the pharmaceutical composition may contain from about 0.5 to 90% by weight of active ingredient. Compositions suitable for topical administration may comprise CTGF in sterile formulation mixed with known suitable ingredients such as paraffin, daserine, cetamol, glycerol and its like to form suitable ointments or creams.

Different pharmaceutical forms and means of delivery may be chosen depending on the precise condition being treated (or prevented etc) and the effect desired to be achieved. Thus, the administration may be acute, for example over a restricted or short period of time, or before, during or after a surgical procedure, or therapeutic intervention e.g. surgery or PCI, or during or after a clinical event such as an acute coronary syndrome, e.g. in unstable angina, or when an acute coronary event (e.g. coronary obstruction) is presented or suspected, or during or immediately after MI, or when MI is suspected or when the subject is at risk of MI, or for the treatment of acute heart failure following an MI. Thus the administration may be acute or short term or transient, i.e. of restricted duration after the heart damaging event. The present invention may thus be seen to provide for the treatment of an acute event, wherein the CTGF may be administered on a short term basis of up to 6 months (e.g. up to 5, 4, 3, 2 or 1 month(s), or less) after the event. Acute, or short term, administration may thus for example be for no longer than 6 months, more particularly no longer than 5, 4, 3, 2 or 1 month(s), or alternatively up to one month, or up to 2, 3, 4, 5 or 6 months. Acute, or short term, administration may also be for shorter time periods, including a single administration, or for up to or no longer than 30, 20, 15, 10, 8, 7, 5, 4, 3, 2 or 1 day(s) or for up to or no longer than 1, 2, 3, 4, 5 or 6 weeks. For such acute administration, CTGF may be administered for example parenterally, e.g. intravenously, for example by intravenous injection, or directly to the heart via a catheter positioned in the coronary artery, e.g. during PCI. In terms of administration of CTGF before a surgical or therapeutic intervention, preferably the CTGF is administered shortly before (e.g. no more than 72, 60, 48, 36 or 24 hours before, preferably not more than 12, 6 or 3 hours before), or immediately before the procedure, or as noted above, it can be administered during or after the procedure

In a preferred embodiment CTGF is administered during or after a heart-damaging event, e.g. an ischaemic event and particularly an acute coronary syndrome. Example 2 below presents the results of an experiment showing beneficial results of CTGF administered post-ischaemia in protecting the heart from the damaging effects of the ischaemia and subsequent reperfusion. In particular, infarct size is reduced. This supports the proposed therapeutic utility in administering CTGF after the heart-damaging event, and in particular after ischaemia, or post-infarction. For example, CTGF may be administered to prevent or reduce myocardial infarction, e.g following or during ischaemia or an ischaemic event, or during or after myocardial infarction, for example to limit or reduce the effects of the infarction.

As mentioned above, CTGF may be administered during or after a heart damaging event, (e g immediately or shortly after, i.e. within 72, 60, 48, 36, 24, 12, 6 or 3 hours after the event) and such administration may be continued for a period of time after the event, for example for a period of up to one year, or up to 9, 6, 5, 4, 3, 2 or 1 month(s). Administration may continue for at least 1 month, or at least 2, 3, 4, 5 or 6 months, or up to 1 month, or up to 2, 3, 4, 5 or 6 months. Such administration may result in an improvement of normalization of cardiac function (for example an improvement as compared with an untreated subject, or as compared to the subject prior to treatment). This improvement or normalization may be maintained after administration has ceased. As discussed above, however, prolonged or long-term administration of CTGF is also included, after the heart-damaging event, on a more “chronic” basis (e.g. for more than one year, or indeed for a number of years, or continuously, or on an on-going basis).

Such effects, or more particularly such therapeutic proposals are supported by the data presented in Example 3 below. This reports the results of a study analysing CTGF levels in the blood (more particularly in the plasma or serum) of patients who have undergone myocardial infarction and PCI. It will be seen that parameters of cardiac function, including in particular ejection fraction are increased, or improved, in patients who have increased serum levels of CTGF following infarction, as compared with patients who have decreased or unchanged levels of CTFG following infarction. In particular, such patients exhibit reduced infarct size, reduced left ventricular dilatation of the heart, and/or improved cardiac function as determined for example by ejection fraction. This suggests that increased or elevated levels of CFTG post-infarction are beneficial in protecting the heart from the effects of the infarction. This supports the proposed therapeutic intervention, to administer CTGF after or during a heart-damaging event, such as ischaemia or an acute coronary syndrome, including particularly after or during MI. Such administration may “mimic” the natural physiological response of the body which may be seen in certain patients, which increases CTGF after an event such as MI. As described in Example 3 below, more particularly the results show that patients with increased CTGF levels for the first two months after the infarction, showed improvement in cardiac function and reduced infarct size. This leads to the proposal, which is in accordance with discussions above, to administer CTGF for a period of at least 2 months, and more particularly for a period of up to 2, 3, 4, 5 or 6 months after the heart-damaging event.

More long term or chronic administration may be required in the treatment of other conditions e.g. chronic conditions, e.g. chronic heart failure. In such situations, CTGF may also be administered intravenously, or via catheter to the heart, or it may be administered orally, or by other means, e.g. by intramuscular or sub-cutaneous injection. For example continuous infusion via a catheter, or intravenously may be required, e.g. via a drip. In such a situation, gene therapy may present an attractive option, to achieve a more long term or sustained delivery of CTGF.

Compositions may also be prepared containing CTGF for use in transportation of an explanted heart, for example to perfuse the heart during transportation or storage or as a storage medium. Such compositions represent a further novel aspect of the present invention.

Thus, in a still further aspect the invention provides a medium for storage or transportation of an isolated heart, said medium comprising CTGF.

Such a medium will be a conventional cardioplegia solution supplemented with CTGF. Cardioplegia solution usually contains NaCl (mM) 0-250, KCl (mM) 0-250, Glucose (mM) 0-200, Insulin (U/1) 0-200, and CaCl₂ (mM) 0-20, and may also contain pyruvate and amino acids. This may also contain impermeants (e.g. mannitol) to reduce intracellular edema. The medium may contain ingredients or additives which may be included to maintain or preserve the heart, such as insulin as noted above.

As noted above, in work leading up to the present invention a transgenic mouse was created which expressed CTGF in the heart i.e. in a cardiac-restricted manner. This represents the first animal model of cardiac-restricted CTGF expression and accordingly represents a further aspect of the present invention.

Thus, the inventors have designed an in vivo system in which the cardioprotective effects of CTGF can be studied. The system is based on an animal model, specifically an animal which expresses CTGF in a cardiac-restricted manner. The animal is thus a transgenic animal, in other words an animal which carries a transgene, i.e. a foreign, “introduced” or heterologous nucleic acid molecule comprising a nucleotide sequence encoding CTGF, i.e. an animal into which such a nucleic acid molecule has been introduced or a progeny of such an animal. The animal thus contains a nucleic acid molecule which is “foreign” or “exogenous” to that animal, and this includes the introduction of a further copy of an endogenous. CTGF gene. The nucleic acid molecule may be any nucleic acid molecule encoding CTGF as defined herein.

The system has the advantage that it is an in vivo rather than in vitro system; the cardioprotective effect of CTGF in the whole animal can be studied, rather than in isolated hearts or cardiac cells alone. The effects of CTGF can be studied in the context of the whole animal; both local and systemic effects can be studied.

The effects of CTGF can be assayed directly in vivo in a non-invasive manner without the need to kill the animal. Thus, the number of animals required for testing is reduced compared with conventional animal experiments.

As indicated above, in a further aspect the invention therefore provides a transgenic non-human animal which expresses CTGF in a cardiac-restricted, or cardiac-specific, manner. The transgenic animal thus contains (or carries) a ‘transgene’, or an introduced (e.g. a heterologous) nucleic acid molecule which comprises a nucleotide sequence encoding CTGF and a promoter that drives cardiac-restricted expression of CTGF. In one embodiment of the invention the promoter employed is that of the α-myosin heavy chain gene; this may comprise the complete intergenic region (5.5 Kb) between the β- and α-myosin heavy chain genes.

By ‘transgenic’ is meant an animal having genetic material artificially introduced or inserted into its genome. In other words, the animal comprises exogenous DNA which has been introduced into the genome (in the sense of DNA “foreign” to that animal; as noted above thus may include a copy of a native or endogenous gene—the point is that nucleic acid material is introduced into the animal). Such an introduced nucleic acid molecule may be viewed as “heterologous”, and in this context “heterologous” is given a broad meaning to include “additional to the genome of the host animal”, as well as “heterologous” in the sense of a nucleic acid molecule (or gene) which does not normally (or “natively”) occur in that animal. This genetic material may be present as an extrachromosomal element or may be stably integrated into the genome in all or a portion of the cells of the animal. Advantageously, the transgenic non-human animal will have stable changes to its germline genomic sequence. The transgenic animal may be homozygous or heterozygous for the genetic alteration. Homozygous animals may be bred using standard techniques from heterozygous animals.

Techniques for the generation of transgenic animals are well known in the art. A recombinant nucleic acid construct which contains a nucleotide sequence encoding CTGF under the control of an appropriate promoter is first generated. This can be introduced into the pronucleus of fertilised eggs according to one widely used technique (Hogan et al. 1994 Manipulating the Mouse Embryo, 2nd Edition Cold Spring Harbor Laboratory Press, Cold Spring Harbor, N.Y.). The construct to be injected into the fertilised egg must first be in the linear form which may readily be achieved, e.g. by digesting the DNA with a suitable restriction endonuclease. The injected, fertilised eggs are then implanted into a foster pseudopregnant mother for the duration of gestation. The offspring are then tested for the presence of the transgene, e.g. using PCR or real-time quantitative PCR. These “founder” animals are then bred, firstly to determine whether the transgene is passed on to the offspring, and then to determine whether or not an offspring animal in fact contains the transgene. The founder animals are then bred to homozygosity. Once the transgenic non-human animals have been bred to homozygosity, continued testing for the presence of the transgene is not necessary.

Alternatively, the recombinant nucleic acid constructs containing the nucleotide sequence encoding the CTGF under the control of an appropriate promoter (for example the α-myosin heavy chain promoter) can be introduced into a pluripotent cell (embryonic stem cell (ES cell)). If this method is used, a sequence encoding a positive selection marker and regions containing sequences that are homologous to the genomic sequences of the animal (regions of homology) are generally also included in the construct.

ES cells are cultured under suitable conditions, and the recombinant targeting constructs are introduced into the ES cells by any method which will permit the introduced molecule to undergo recombination at its regions of homology, for example, micro-injection, calcium phosphate transformation, or electroporation (Toneguzzo, F. et al., Nucleic Acids Res. 16: 5515-5532 (1988); Quillet, A. et al., J. Immunol. 141: 17-20 (1988); Machy, P. et al., Proc. Natl. Acad, Sci. (U.S.A.) 85: 8027-8031 (1988)).

The construct to be inserted into the ES cell must first be in the linear form, which may be achieved e.g. by digesting the DNA with a suitable restriction endonuclease. After introduction of the genetic sequences, the ES cells are cultured under conventional conditions and screened for the presence of the construct using known techniques. Cells that survive the selection process are then screened by other methods, such as PCR or real-time quantitative PCR, for the presence of integrated sequences.

The selected ES cells containing the construct in the proper location are identified, are inserted into an embryo, preferably a blastocyst, for example by microinjection. The appropriate stage of development of the embryo at which the ES cells are inserted depends on the particular species that is used for generation of the transgenic non-human animal. In mice it is about 3.5 days.

After the ES cell has been introduced into the blastocyst, the blastocyst is typically implanted into the uterus of a pseudopregnant foster mother for gestation. Offspring are then screened using standard techniques known in the art e.g. Southern blots and/or PCR. Mosaic (chimeric) offspring are then bred to each other to generate homozygous animals.

As is the case for transgenic animals generated by pronuclear injection, homozygotes and heterozygotes may be identified e.g. by Southern blotting of equivalent amounts of genomic DNA from animals that are the product of this cross, or with known heterozygotes or wild type animals.

The transgenic animal may be any non-human animal, but is preferably a mammal and more preferably a domestic or livestock animal such as a cow, pig, goat, sheep, horse or fanned fish or a laboratory animal e.g. a primate or a rodent such as rat, mouse, hamster, rabbit or guinea pig. Preferably the transgenic animal is a rodent and most preferably a mouse.

The transgenic non-human animal can express CTGF. The transgene may be any nucleic acid molecule encoding CTGF as defined herein. In certain embodiments, the transgenic non-human animal can overexpress CTGF in the heart compared with the hearts of non-transgenic animals by 10, 20, 30, 40, 50, 60, 70, 80, 90 or 100 fold. Measurement of the level of expression can be achieved by techniques in the art, for example PCR or Western blot.

The transgenic non-human animal can be used to assay or determine the cardioprotective effects of CTGF. As such, there is also provided the use of the transgenic non-human animal as described and defined herein in assessing or determining the cardioprotective effects of CTGF.

In a further embodiment of the invention, there is provided a method of making a transgenic non-human animal of the invention, said method comprising the step of introducing into said animal (more particularly, into the genome of said animal) a nucleic acid molecule comprising a nucleotide sequence encoding CTGF. Such a method may optionally further include the step of crossing such an animal with another animal or breeding progeny from such an animal.

The method may thus comprise the steps of introducing a recombinant genetic construct comprising a nucleic acid molecule encoding CTGF under the control of a cardiac-specific promoter (for example the α-myosin heavy chain promoter) into the pronucleus of a fertilised egg, and implanting said egg into a psuedopregnant foster mother.

Alternatively the method may comprise the steps of introducing a recombinant genetic construct comprising a nucleic acid molecule encoding CTGF under the control of a cardiac-specific (for example the α-myosin heavy chain promoter) promoter into an ES cell, introducing said ES cell into a blastocyst and implanting said blastocyst into a pseudopregnant, foster mother. In this method, it is possible to screen the ES cells for homologous integration of the construct, in which case a positive selection marker should be included in the genetic construct.

In such methods, it would be a matter of routine to select a suitable or desired promoter or an appropriate pseudopregnant foster mother or appropriate ES cell.

The recombinant genetic constructs that are used to generate the transgenic non-human animals will generally include the following components: a nucleic acid molecule comprising a nucleotide sequence encoding CTGF, and a cardiac-specific promoter operably linked to the CTGF. If the transgenic non-human animal is to be made using homologous recombination in ES cells, it is necessary also to include a sequence encoding a positive selection marker, and homologous insertion sequences (Capecchi M R, Trends in Genetics, 1989 5(3):70-6). Insulator sequences, as described in U.S. Pat. No. 5,610,053 can also be included.

Positive selection markers include any gene which encodes a product that can be assayed. Commonly used examples include the hprt gene (Littlefield, J. W., Science 145: 709-710 (1964)) and the TK gene of herpes simplex virus (Giphart-Gassler, M. et al., Mutat. Res. 214: 223-232 (1989)) or other genes which confer resistance to amino acid or nucleoside analogues, or antibiotics. Addition of the appropriate substrate of the positive selection marker can be used to determine if the product of the positive selection marker is expressed.

The expression of the CTGF may be regulated temporally, so that expression only occurs at a particular time during the development of the transgenic non human animal, or else at one or more particular times during the life of the transgenic non human animal. The transgenic non-human animal of the invention can be used to study or determine the effects of CTGF on the post-natal heart, for example the cardioprotective effects.

Expression of the CTGF can also be inducible, in other words the expression of the CTGF can be switched on or off, depending on the local conditions in the cell. These conditions can be manipulated artificially, e.g. by addition of inducer molecules to the transgenic non-human animal. In one embodiment of the invention the CTGF expression may be induced, or switched on, after damage to the heart has occurred, and thus the transgenic non-human animal can be used to study the cardioprotective effect of CTGF after damage to the heart has occurred. This model can be used to determine the therapeutic cardioprotective effects of CTGF on a damaged heart. In one embodiment of the invention damage to the heart is induced, then CTGF expression is switched on or induced to enable the study of the cardioprotective and/or therapeutic effects of CTGF. In further embodiments of the invention the CTGF expression or overexpression can be switched on or off, or induced or terminated before damage to the heart occurs or when damage is occurring or during damage. The prophylactic effects of CTGF can be studied or determined.

To achieve such controlled, or regulated expression, a suitable promoter must be selected e.g. an inducible promoter, or a promoter which is active at certain developmental times or time points. The expression pattern of the CTGF therefore depends on the choice of promoter for generating the transgenic animal. A large number of different promoters are known which can be used to drive expression of CTGF and it is simply the case of using an appropriate promoter to make the genetic construct which is then used to generate the transgenic non-human animal. The most commonly employed promoter that drives cardiac-restricted expression is the α-myosin heavy chain promoter. This promoter has highest activity in the heart after birth and fairly low activities in the fetal heart. The α-myosin heavy chain promoter drives constitutive expression in postnatal life. However, technology has also been developed for inducible expression of a protein in the heart. In order to establish transgenic mice with inducible, cardiac-restricted-overexpression of CTGF, transgenic hybrids of two transgenic lines are generated. Briefly, one of the transgenic lines (Tg-rtTA) expresses a genetically engineered transactivator (rtTA) controlled by tetracycline (or doxycycline). The transactivator rtTA (transcription factor) binds and activates a tetracycline response (enhancer) element (TRE) in the presence of tetracycline (or doxycycline). Expression of rtTA is controlled by the α-myosin heavy chain promoter (α-MHC) to secure cardiac-restricted expression. The other transgenic line (Tg-(CMV)-CTGF) expresses CTGF under control of a minimal CMV (cytomegalovirus) promoter containing the prokaryotic TRE enhancer element. This promoter will be activated by binding of rtTA in the presence of tetracycline (or doxycycline). Thus, transgenic hybrids of these two transgenic lines will allow CTGF to be induced in the heart upon administration of tetracycline or doxycycline to the animal. Usually, tetracycline or doxycycline is included in the drinking water (1 μg/ml). The extent of CTGF expression can also be controlled by the dose of tetracycline or doxycycline. Control mice are usually provided by making hybrids of Tg-rtTA and wild type mice of same genetic background. These mice express the rtTa transcription factor, but do not synthesize CTGF when given tetracycline or doxycycline. This technology for inducible, tissue-specific expression of a protein has previously been described in the literature (Furth, P. A., St Onge, L., Böger, H., Gruss, P., Gossen, M., Kistner, A., Bujard, H., and Hennighausen, L. Proc Natl Acad Sci USA. 91: 9302-9306, 1994, and Kistner, A., Gossen, M., Zimmermann, F., Jerecic, J., Ullmer, C., Lübbert, H., and Bujard, H Proc Natl Acad Sci USA 93, 10933-10938, 1996).

Inducible promoters have the advantage that they can be activated or induced to express CTGF. The CTGF is thus only expressed when it is necessary for the purposes of the experiment. Examples of inducible promoters are well known in the art and include Cre-, estrogen-, retinoic acid responsive element containing promoters and tetracycline responsive promoters (for a review see Albanese C, Hulit J, Sakamaki T, Pestell R G. Semin Cell Dev Biol. 2002 April; 13(2):129-41). Promoter systems may be modified to use such or other response elements, analogously to the manner described above for the tetracycline/doxycycline inducible promoter system.

The invention will now be further described in the following non-limiting Examples, with reference to the following drawings in which:

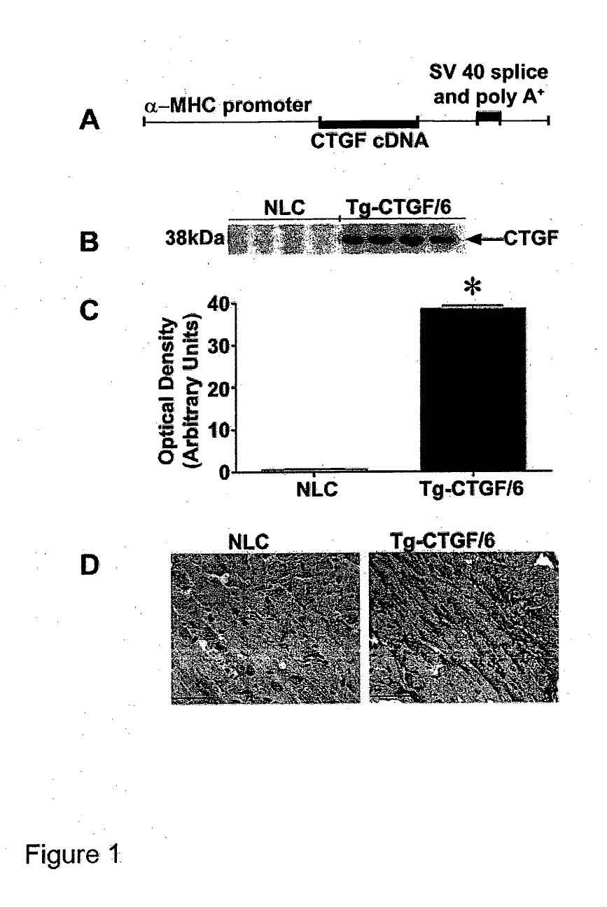

FIG. 1 shows generation of Tg-CTGF transgenic mice. A, Schematic of the CTGF transgene that was constructed with the α-MHC mouse promoter. B, Western blot analysis of myocardial CTGF of NLC and Tg-CTGF mice, showing specific immunoreactive band at 38-kD representing CTGF. C, Densitometric analysis of the immunoreactive bands. Values are mean±SEM, n=4 each group. *P<0.05 vs. NLC group. D, Representative photomicrographs of immunohistochemical staining of CTGF in myocardial tissue sections (6 μm) of NLC and Tg-CTGF mice. Strong anti-CTGF immunoreactivity in myocardial tissue section of Tg-CTGF mouse was restricted to cardiomyocytes.

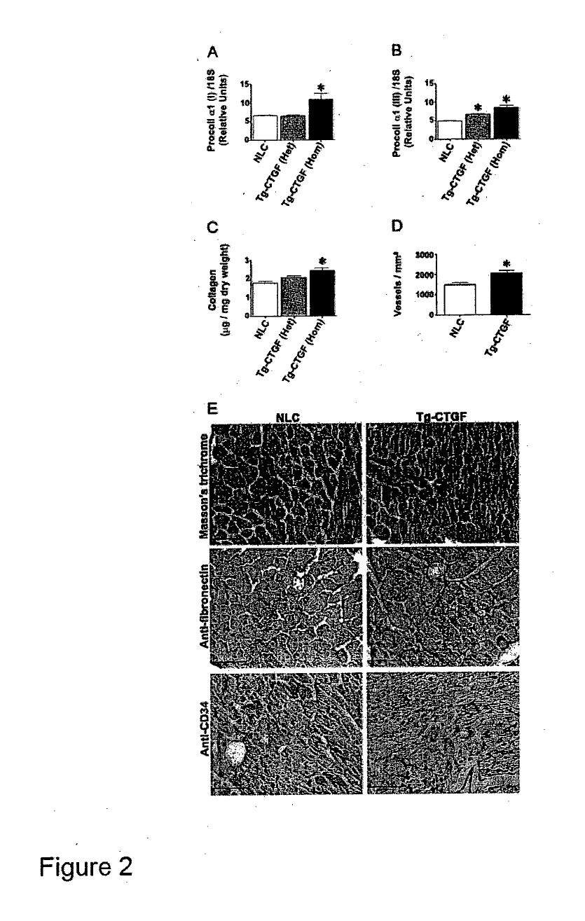

FIG. 2 shows Tg-CTGF mice develop only mild fibrotic changes. A, Myocardial mRNA levels of procollagen α1(I) and B, procollagen α1 (III) of Tg-CTGF and NLC mice. Total RNA of myocardial tissue samples was extracted and mRNA levels were analyzed by quantitative real-time RT-PCR. The data in the histograms are ratios of indicated mRNA relative to 18S rRNA levels. C, Hydroxyproline concentrations (pmol/mg tissue dry weight) in myocardium of Tg-CTGF and NLC mice. Samples (50 mg) of cardiac tissue were lyophilized to constant dry weight, and analysis of hydroxyproline contents was performed by HPLC as detailed in ‘Materials and Methods’. The values are mean±SEM of each group for Tg-CTGF mice (Heterozygous mice, n=8; Homozygous mice, n=8) and NLC mice (n=8). *P<0.05 vs. NLC group. D, Numbers of CD34-positive capillaries in myocardium of Tg-CTGF mice and NLC mice. Results are expressed as mean±SEM of each group for Tg-CTGF mice (n=5) and NLC mice (n=5). *P<0.05 vs. NLC mice. E, Representative photomicrographs of myocardial tissue sections of Tg-CTGF and NLC mice stained with Masson's trichrome (upper two panels) and immunostained with anti-fibronectin antibody (middle two panels) and anti-CD34 antibody (bottom two panels). Magnification: ×400.

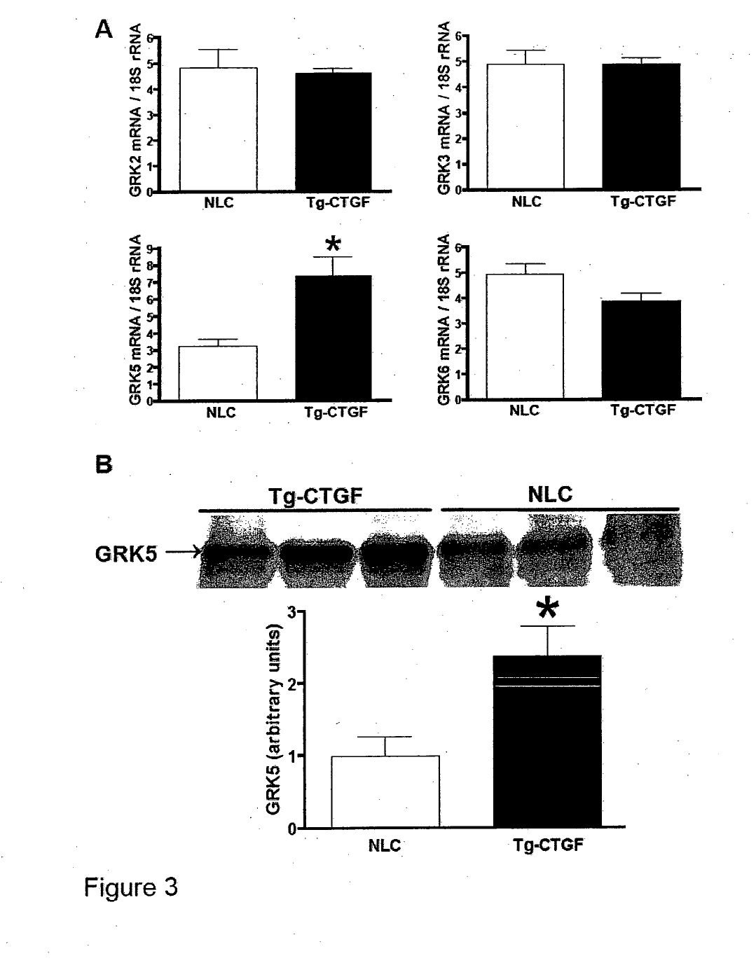

FIG. 3 shows expression of GRK isoforms in cardiac myocytes from Tg-CTGF mice and nontransgenic control mice. A, Real-time quantitative PCR of mRNA levels of GRK2, GRK3, GRK5, and GRK6 in cardiac myoctes from Tg-CTGF mice (closed bars) and from nontransgenic control mice (open bars). MGB probes and primers for mouse GRK2, GRK3, GRK5 and GRK6 were from Applied Biosystems. mRNA levels are presented relative to 18S RNA levels in the cardiac myocyte samples. Results are expressed as mean±SEM of data from cardiac myocytes from Tg-CTGF mice (n=4) and nontransgenic control mice (n=4). *P<0.05 vs. NTC mice. B, Immunoblot of GRK5 immunoprecipitated from extracts of cardiac myocytes from Tg-CTGF mice (n=3) and nontransgenic control mice (n=3) confirming upregulation of GRK5 in Tg-CTGF mice. Positive control in extracts from Sf9 cell expressing recombinant CTGF. Histogram demonstrates data from densitometric scanning of immunoreactive GRK5 on immunoblot.

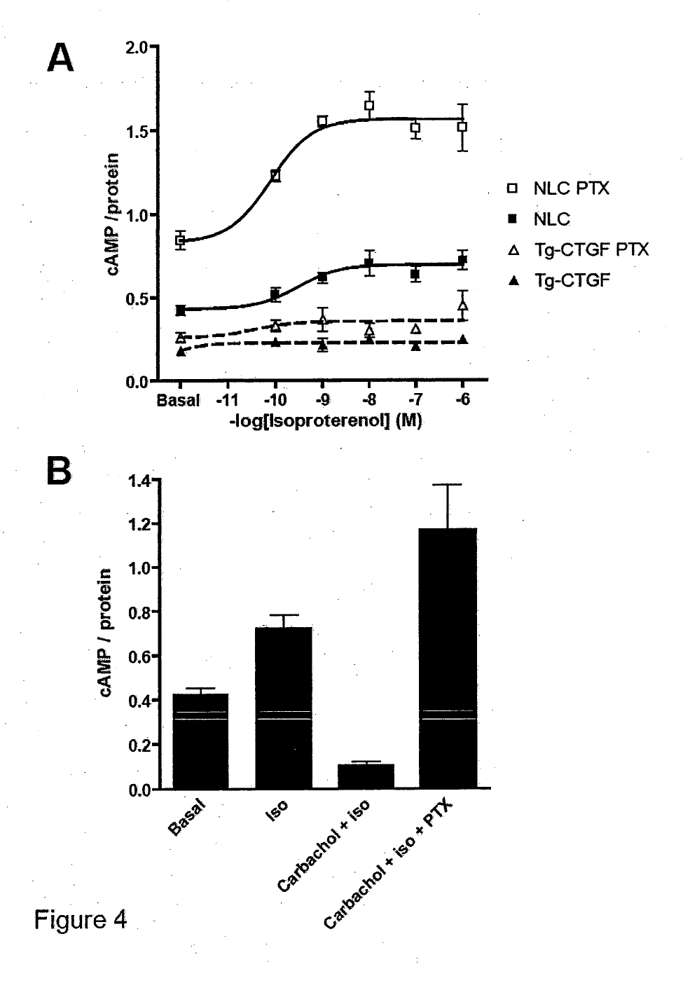

FIG. 4 shows concentration-effect curves of isoproterenol-stimulated cAMP generation in cardiac myocytes from Tg-CTGF mice and nontransgenic control mice. Cardiac myocyte were pretreated (10 min) with 0.5 mmol/l 3-isobutyl-1 methylxanthine (phosphodiesterase inhibitor) prior to start of assay by addition of isoproterenol. Data are mean±S.D of cAMP levels in cardiac myocytes from triplicate wells from Tg-CTGF mice and nontransgenic control mice for each given concentration. The experiment is representative of 3 independent experiments.

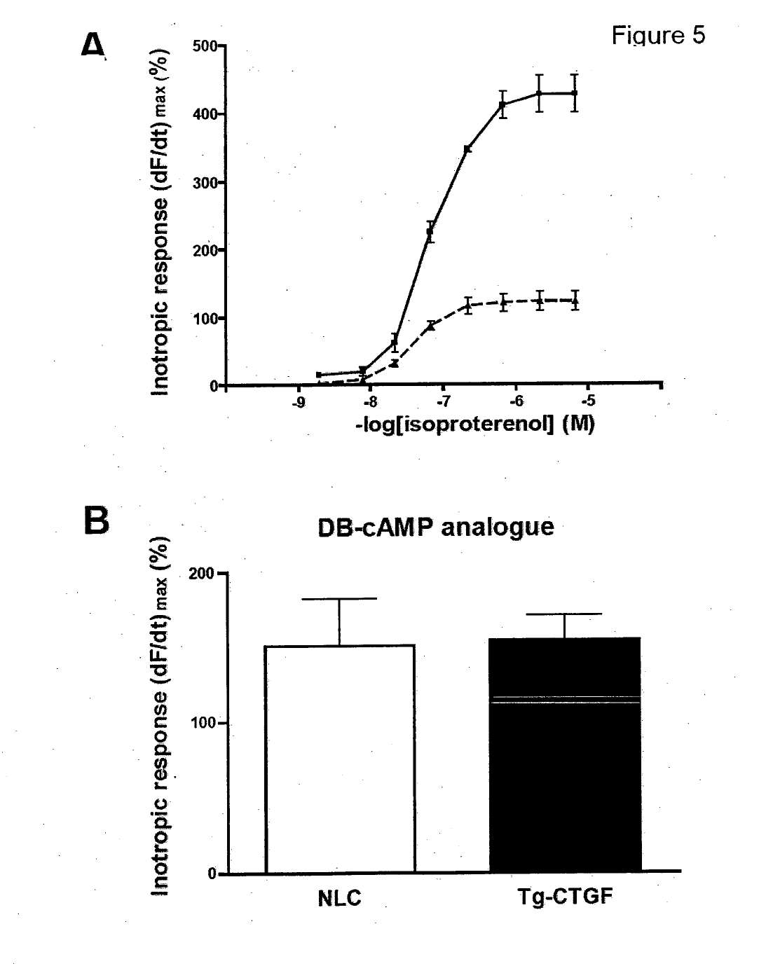

FIG. 5 shows concentration-effect curves of isoproterenol-stimulated contractility of isolated papillary muscles from Tg-CTGF and non-transgenic control (NLC) mice. A, semi-logarithmic plot of isoproterenol-stimulated maximal inotropic responses expressed as increase in (dF/dt)max as percent above basal (non-stimulated) (dF/dt)max. The data are mean±S.E. of independent observations from Tg-CTGF (n=6) and NLC (n=6) mice. B, Maximal inotropic responses in isolated papillary muscle strips from Tg-CTGF mice (n=6) and NLC mice (n=6) stimulated with a supramaximal concentration of dibutyryl-cAMP (10 mmol/l). Data are presented as % increase above maximal contractility (dF/dt max) prior to stimulation with dibutyryl-cAMP (mean±S.E.).

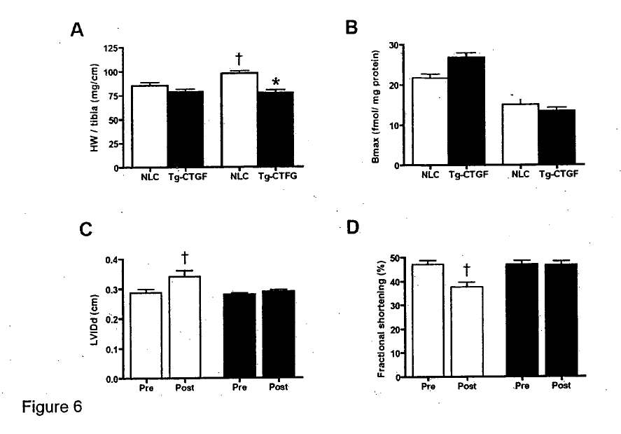

FIG. 6 shows β-adrenergic receptor-induced cardiomyopathy in Tg-CTGF mice versus non-transgenic control mice (NLC). Tg-CTGF mice (n=9) and NLC mice (n=9) were subjected to continuous treatment with isoproterenol (150 mg/kg/day by subcutaneous delivery via micro-osmotic pumps) for 14 days. Similar groups of Tg-CTGF mice (n=9) and NLC mice (n=9) that received vehicle (saline) were included as controls. A, Cardiac mass at end-point immediately following termination of treatment protocol. Cardiac mass is presented as heart weight relative to tibia length. B, β-adrenergic receptor densities in membranes from myocardial tissue sampled immediately after termination of treatment protocol. β-adrenergic receptor densities were determined by radioligand binding assay using [¹²⁵I]-iodocyanopindolol as detailed in Material and Methods. C and D, Left-ventricular-end diastolic diameter (LViDd) and fractional shortening (FS) determined by transthoracic echocardiography at study end-point. All data is presented as mean±S.E.M. for the indicated treatment groups.

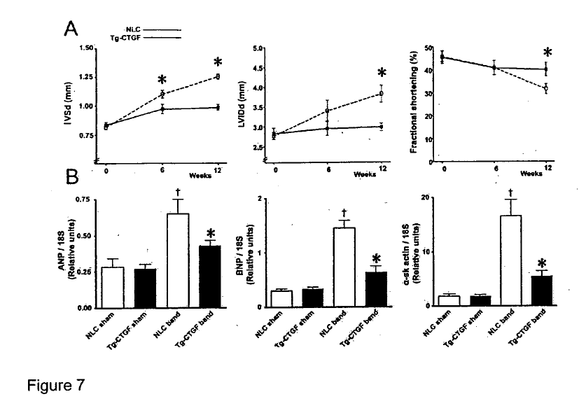

FIG. 7 shows A, Serial echocardiographic measurements of Tg-CTGF (n=9) and NLC (n=9) mice at baseline 6 and 12 weeks after induction of pressure overload. B, Quantitative real-time RT-PCR analyses of myocardial mRNA levels of ANP, BNP and α-skeletal actin of Tg-CTGF and NLC mice. The data in the histograms are ratios of indicated mRNA relative to 18S rRNA levels. The values are mean±SEM of each group for Tg-CTGF mice (Tg-CTGF sham, n=7; Tg-CTGF band, n=9) and NLC mice (NLC sham, n=6; NLC band, n=9). †P<0.05 vs. NLC sham; *P<0.05 vs. NLC band.

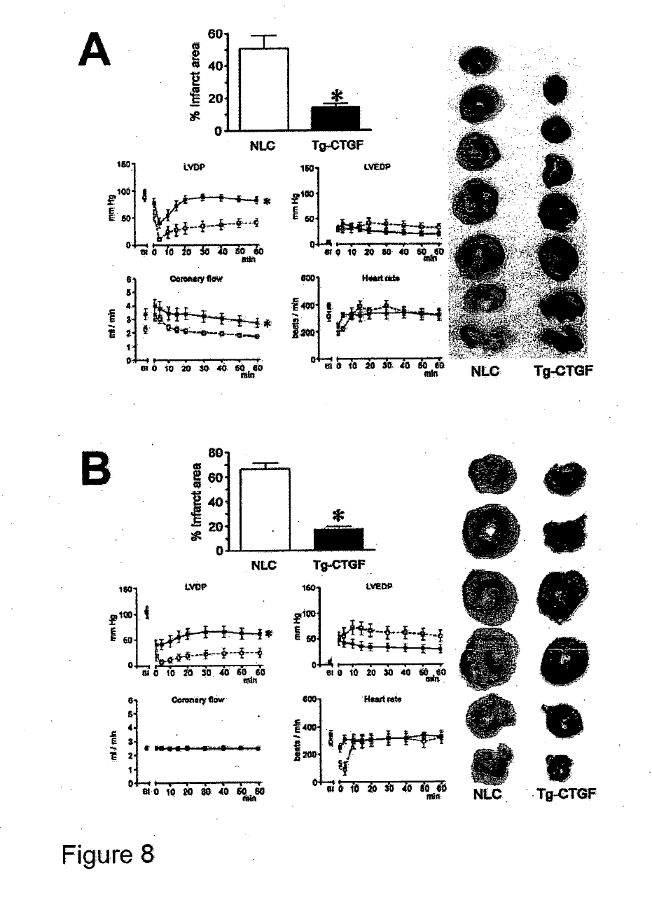

FIG. 8 shows isolated, Langendorff-perfused Tg-CTGF hearts displayed markedly reduced infarct size after 40 min of ischemia and 60 min of reperfusion as compared to non-transgenic control hearts (NLC hearts), both under conditions with constant perfusion pressure (panel A) and constant coronary flow (panel B). Recovery of cardiac function after ischemia, as measured by left ventricular developed pressure, was enhanced in Tg-CTGF hearts in both experiments. Importantly, no significant differences in heart rate were detected. Data are mean±SE of Tg-CTGF hearts (n=8) and non-transgenic control hearts (n=8). Data were analyzed by two-way ANOVA, and post-hoc analysis was performed with Bonferroni's t-test accounting for multiple comparisons. *P<0.05 vs. NLC hearts.

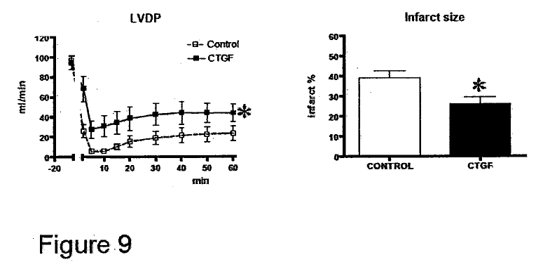

FIG. 9 shows isolated mouse hearts subjected to Langendorff-perfusion with Krebs-Henseleit solution with or without recombinant human CTGF prior to ischemia and reperfusion. Mouse hearts were perfused with Krebs-Henseleit solution in the absence (□) or presence (▪) of recombinant human CTGF (75 nmol/l) for 10 min, then subjected to 40 min of ischemia, and finally reperfusion for 60 min. Reperfusion was performed with Krebs Henseleit without any additions. Data are mean±SE of Tg-CTGF hearts (n=8) and non-transgenic control hearts (n=8). Data were analyzed by two-way ANOVA, and post-hoc analysis was performed with Bonferroni's t-test accounting for multiple comparisons. *P<0.05 vs. NLC hearts. Infarct size was determined by TTC (triphenyltetrazolium chloride)-staining of myocardial sections after completion of the reperfusion protocol with subsequent determination of relative segmental area undergoing infarction. Data are mean±SEM of n=8 in each group. *P<0.05 vs. non-treated hearts.

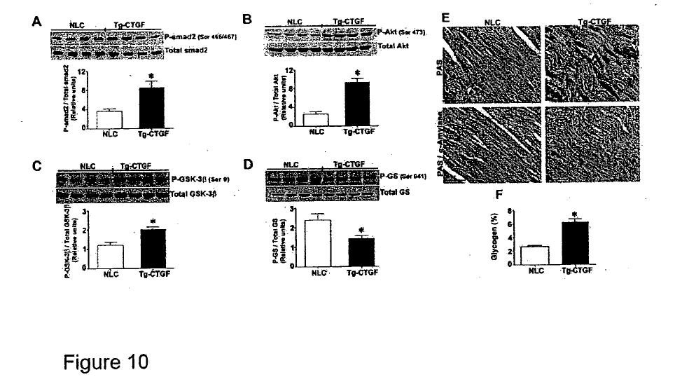

FIG. 10 shows activation of Smad2 and Akt/GSK-3β signaling pathways in Tg-CTGF mice heart. A, Representative Western blots for the cardiac level of phosphorylated Smad2 (P-Smad2) and total Smad2 in transgenic mice and NLC mice. B, Representative Western blots for the cardiac level of phosphorylated Akt (P-Akt) and total Akt in transgenic mice and NLC mice. C, Representative Western blots for the cardiac level of phosphorylated GSK-3β (P-GSK-3β) and total GSK-3β in transgenic mice and NLC mice. D, Representative Western blots for the cardiac level of phosphorylated GS (P-GS) and total GS in transgenic mice and NLC mice. The intensities of bands were measured by densitometric scanning of the autoradiograms. The data in the histograms are ratios of indicated phospho-protein relative to respective total protein levels. Values are mean±SEM, n=4 each group. *P<0.05 vs. NLC group. E, Representative Periodic Acid-Schiff (PAS) staining of NLC and Tg-CTGF mice (upper two panels). Pretreatment with α-amylase confirmed the specificity of staining (bottom two panels). Magnification: ×400. F, Quantification of glycogen contents. Glycogen contents in hearts from Tg-CTGF and NLC mice was measured as described in ‘Materials and Methods’. Results are expressed as mean±SEM of each group for Tg-CTGF mice (n=8) and NLC mice (n=7). *P<0.05 vs. NLC mice.

FIG. 11 shows the alignment of predicted peptide sequences of human, rat and mouse CTGF. Boxes indicates amino acid residues conserved among the three species.

FIG. 12 shows the nucleotide sequence of human CTGF

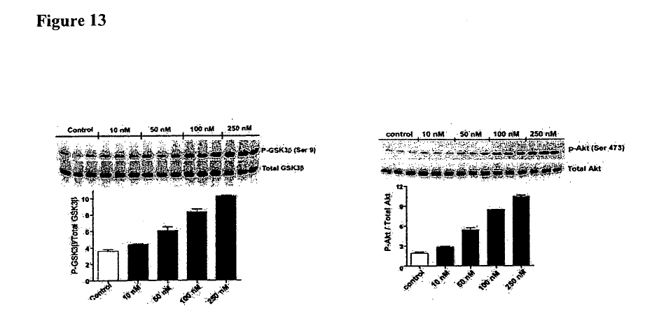

FIG. 13 shows cardiac myocytes stimulated with increasing concentrations of recombinant human CTGF for 30 min at 37° C. Cells were subsequently harvested in RIPA buffer in the presence of phosphatase inhibitor (sodium orthovanadate and sodium fluoride), denatured in Laemmli buffer and subjected to polyacrylamide gel electrophoresis. Proteins were transferred by electroblotting onto PVDF membranes and subjected to immunostaining with anti-phospho(ser473)-Akt and anti-phospho(ser9)-GSK-3β—specific antibodies and secondary HRP-conjugated anti-rabbit IgG. FIG. 13 shows Western blot analysis of extracts of adult mouse cardiac myocytes stimulated with increasing concentrations of CTGF

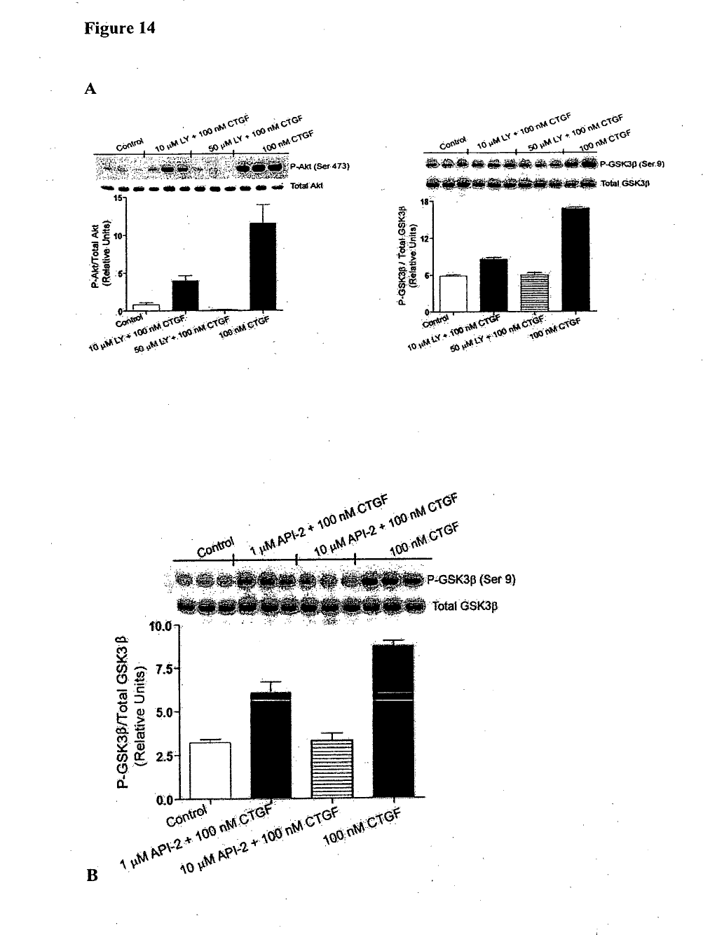

FIG. 14 shows cardiac myocytes stimulated with recombinant human CTGF (200 μmol/L) for 30 min at 37° C. in the presence or absence of (A) phosphoinositide-3 kinase inhibitor (LY294002; 50 μmol/L) or (B) Akt-inhibitor (API-2; 10 μmol/L). Cells were subsequently harvested in RIPA buffer in the presence of phosphatase inhibitor (sodium orthovanadate and sodium fluoride), denatured in Laemmli buffer and subjected to polyacrylamide gel electrophoresis. Proteins were transferred by electroblotting onto PVDF membranes and subjected to immunostaining with anti-phospho(ser9)-GSK-3β—specific antibody and secondary HRP-conjugated anti-rabbit IgG. Panel A shows adult cardiac myocytes preincubated with PI3K-inhibitor (LY294002) and subsequently stimulated with CTGF. Panel B shows adult cardiac myocytes preincubated with AKT inhibitor (API-2) and subsequently stimulated with CTGF.

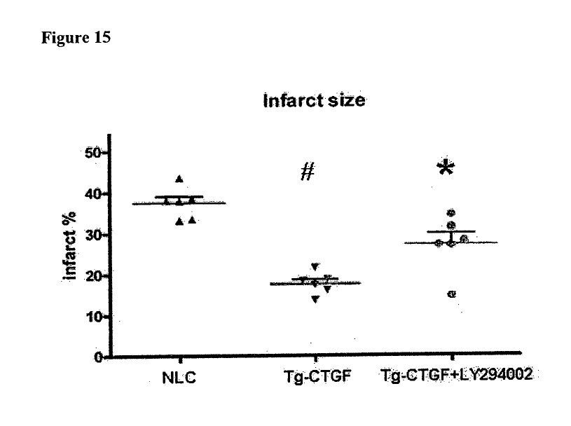

FIG. 15 shows hearts from wild-type and Tg-CTGF mice subjected to perfusion ex vivo in Krebs-Henseleit buffer ad modum Langendorff. As indicated in FIG. 15, Tg-CTGF mice were pretreated with phosphoinositide inhibitor LY294002 (15 μmol/kg/day s.c.; n=6 or DMSO vehicle; n=6) by subcutaneous injection for 5 days to inhibit CTGF-stimulated, PI3 kinase-dependent phosphorylation and activation of Akt and compared with hearts from non-transgenic control mice (NLC; n=6). Hearts were subjected to 40 min of no-flow ischemia and subsequent reperfusion for 60 min in Krebs Henseleit buffer. Infarct size were subsequently assessed by 2,3,5-triphenyltetrazolium chloride (TTC)-staining of remaining viable tissue in serial segments of the left ventricle and digital scanning analysis. *P<0.05 vs. Tg-CTGF hearts. #P<0.05 vs NLC hearts.

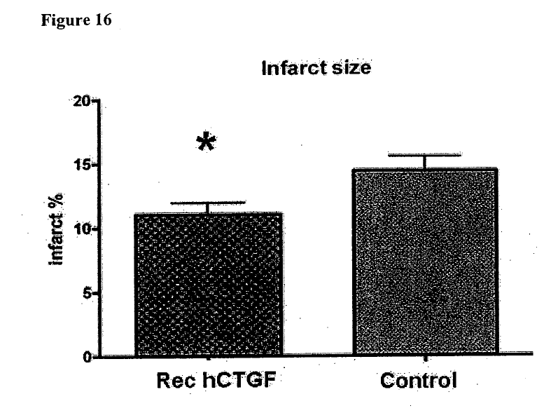

FIG. 16 shows C57/BL6 (wild type) mice that were subjected to Langendorff-perfusion in Krebs Henseleit buffer ex vivo. Hearts were subjected to 40 min of global ischemia and subsequently reperfusion in Krebs Henseleit buffer in the presence (n=6) or absence (n=6) of recombinant human CTGF (100 nmol/L). Hearts were exposed to CTGF during the first 10 min of reperfusion. Reperfusion was continued in Krebs Henseleit buffer for a total of 60 min before experiments were terminated and myocardial infarct size were determined by TTC-staining of serial segments of the left ventricle. FIG. 16 shows post-ischemic coronary perfusion of the heart with recombinant hCTGF reveals sustained cardioprotective action. *P<0.05 vs. control group.

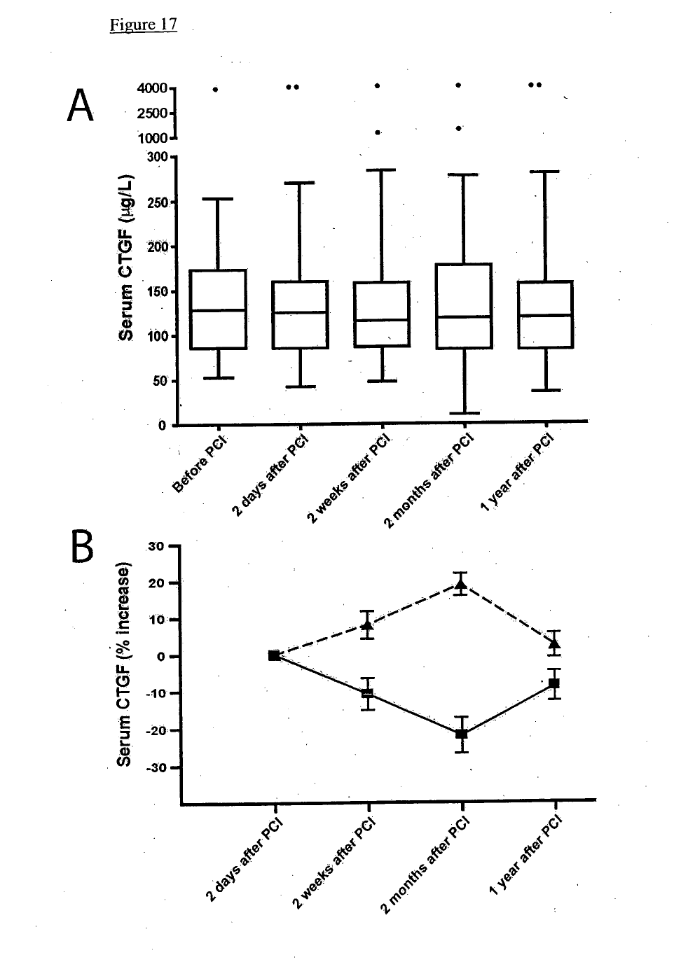

FIG. 17 Panel A shows mean plasma CTGF levels in patient cohort immediately before PCI, 2 days and 14 days after PCI, 2 months after PCI and 1 year after PCI (N=49 patients.). Panel B shows plasma CTGF levels in patient cohort stratified according to those patients who demonstrated elevations of plasma CTGF level after the acute ischemic event versus those that demonstrated lowering of plasma CTGF levels after the index event. Similar number of patient stratified to the two groups.

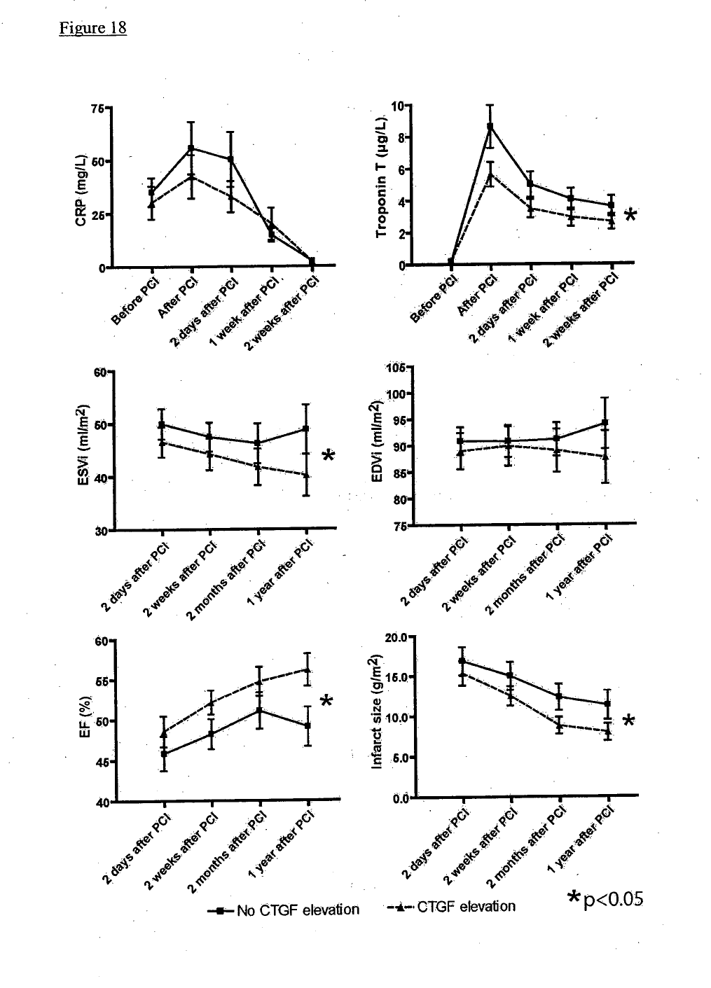

FIG. 18 Upper panels demonstrate plasma C-reactive protein (CRP) and Troponin T in blood samples drawn immediately before PCI and at follow up 2, 7 and 14 days after PCI. Middle and lower panels demonstrate data from functional MRI imaging of the heart 2 days, 14 days, 2 months and 1 year after PCI. The data are end-systolic and end-diastolic volume index, ejection fraction and infarct size. The latter is determined after administration of gadolinium contrast agent. P<0.05 for group difference (patients with elevated plasma CTGF levels versus patients with decreasing CTGF levels after index event) determined by 2-way analysis of variance.

### EXAMPLES

Materials and Methods

Generation of Transgenic CTGF Mice

A DNA fragment encoding the entire ORF of rat CTGF cDNA (GenBank accession. No. NM022266) under control of the mouse α-myosin heavy chain (α-MHC) promoter was constructed as shown schematically in FIG. 1A. The CTGF cDNA was preceded by the Kozak consensus sequence for initiation of transcription, and flanked at the 3′-end by SV40 splice and polyA⁺ signals. Transgenic mice were generated by pronuclear injection of the linearized DNA construct into fertilized oocytes from C56BL/6-CBA mice and subsequent implantation of the oocytes in pseudopregnant mice. Incorporation of transgene into the genome of the resultant offspring was confirmed by real-time quantitative PCR for detection of the SV40 DNA sequence in genomic DNA. Sequence-specific PCR primers (forward, 5′-CAGTGGTGGAATGCCTTTAATGA-3′(SEQ ID NO:7); reverse, 5′-AGGAGTAGAATGTTGAGAGTCAGCAGTA-3′ (SEQ ID NO:8)) and TaqMan probe (probe, FAM-CTCAGAAGAAATGCCATCTA-MGB (SEQ ID NO:9)) for the SV40 DNA were designed using the Primer Express software version 1.5 (Applied Biosystems). Two transgenic founder lines, i.e. Tg-CTGF/6 and Tg-CTGF/13 (demonstrating highest myocardial expression of CTGF by Western blot analysis) were established and propagated. The founder mice (FO) were backbred with C56BL/6 inbred mice to generate the F1 generation. Further expansion was performed by mating transgenic siblings within the F1 generation. Non-transgenic littermate controls (NLC) were generated from non-injected C56BL/6-CBA mice (siblings of the mice employed for pronuclear injection), and bred similar to the transgenic lines, i.e. similar background as Tg-CTGF mice. Unless otherwise indicated, mice of the Tg-CTGF/6 line were employed in all experiments.

Isolation of Cardiac Myocytes and Determination of Cardiac Myocytes Surface Area

Cardiac myocytes were isolated from Tg-CTGF and NLC hearts (male, 3 months) by Ca²⁺-free retrograde perfusion and enzymatic digestion as previously described in O'Connell et al 2007 Methods Mol Biol 357:271-296. Isolated cardiac myocytes were plated in wells pre-coated with mouse laminin (Invitrogen Inc.) and maintained in Minimum Essential Medium (MEM) with Hanks' salts supplemented 10 mmol/l 2,3-butanedionemonoxime, 0.1 mg/ml bovine serum albumin, 0.1 umol/l insulin, and 0.1 nmol/l thyroxin in humidified atmosphere containing 5% CO2.

RNA Isolation, Microarray Analysis and Real-Time Quantitative Reverse-Transcription Polymerase Chain Reaction

Total RNA was isolated from myocardial tissue samples by homogenization in chaotropic salts and subsequent ion exchange chromatography using the RNeasy system (Qiagen, Germany). Myocardial RNA from Tg-CTGF (n=4) and NLC (n=4) mice (male; 4 months old) was analyzed by Affymetrix GeneChip Expression Arrays (Mouse Genome 430A 2.0 GeneChip Arrays. Hybridization signals of myocardial RNA from Tg-CTGF mice and NLC mice were filtered and analyzed using the robust multichip analysis algorithm (RAM) of the genes that remained confidently identified after filtering (14072 genes). For real-time quantitative PCR, total RNA was reverse transcribed by using TaqMan Reverse Transcription Reagents Kit, and subsequently real-time quantitative PCR of each sample was run in triplicates using TaqMan Pre-Developed Assay Reagents and the ABI Prism 7900 Sequence Detection System and software (Applied Biosystems, Foster City, Calif., USA) according to the manufacturer's instructions: A standard curve was obtained by amplifications of cDNA obtained from serial dilutions of myocardial total RNA. For all specific mRNA amplified linear inverse correlations were observed between amount of mRNA and CT value (number of cycles at threshold lines). Gene expression was presented relative to the levels of 18S rRNA as the housekeeping gene.

Immunoprecipitation of GRK5

Immunodetection of myocardial levels of GRK5 in NLC and Tg-CTGF mice was performed in isolated cardiac myocytes followed by immunoprecipitation. The isolated cardiac myocytes were solubilised in RIPA-buffer (0.15M NaCL, 10 mM Tris-HCl pH 7.3, 0.5% NP-40, 5 mM EDTA, 0.2 mM PMSF, 1 ug/ml aprotinin, 1 ug/ml pepstatin and 1 ug/ml leupeptin) for 30 min at 4° C., and subsequently clarified by centrifugation for 10 min at 3000 rpm. GRK5 was immunoprecipitated from clarified extract with of anti-GRK 4-6 IgGi (clone A16/17, Upstate Biotechnology, Inc.) overnight at 4° C. Capturing of the immunocomplex was performed with 100 uL of 50% Protein A agarose bead slurry, agitated for 2 h at 4° C. Immune complexes were washed 2 times with PBS and the agarose beads were resuspended in 2× loading buffer before protein-gel loading.

Western Blot Analysis

Total protein extracts of myocardial tissue samples were separated by SDS-polyacrylamide gel electrophoresis (SDS-PAGE) and transferred to PVDF membrane by electroblotting. The membranes were incubated with anti-CTGF IgG, anti-GRK5 IgG (H-64:sc-1 1396, Santa Cruz Biotechnology), or antibodies against smad2, phospho-smad2 (Ser 465/467), Akt, phospho-Akt (Ser 473), GSK-3P, phospho-GSK-3p (Ser 9), GS or phospho-GS (Ser 641) antibodies (Cell Signaling Technologies). Immunoreactivity was detected using an enhanced chemiluminescence reaction system according to the manufacturer's instructions (ECL, Amersham Biosciences).

Assay of Receptor-Generated Responses in Isolated Cardiac Myocytes

Six hours before assay of receptor-generated responses, the cell culture medium was replaced with MEM containing the supplements indicated above except insulin and thyroxin. The cells were subsequently stimulated with isoproterenol; 15 min, in the presence of IBMX (0.1 mM). The assays were stopped at the indicated time points and harvested in sample buffer (50 mmol/l Tris-HCl, pH 6.8, 10% glycerol, 4% sodium dodecylsulphate, 1 mmol/l sodium orthovanadate, 5 mmol/l EDTA, and 1 mmol/l phenylmethanesulfonyl fluoride) for Western blotting. The cell lysates were separated by SDS-PAGE and electroblotted onto PVDF membranes. The filter membranes were subjected to immunoblot analysis with anti-phospho-ERK1/2-specific IgG (anti-phosphothreonine-202/phosphotyrosine-204 ERK1/2, Cell Signaling Technology Inc.) or anti-phosphoserine 16 phospholamban IgG (Upstate Biotechnology) according to the manufacturer's instructions. To confirm similar levels of total ERK1/2 and total phospholamban parallel filter membranes were subjected to immunoblot analysis with anti-ERK1/2 IgG (Cell Signaling Technologies, Inc.) or anti-phospholamban IgG (Upstate Biotechnology) according to the manufacturer's instructions.

Assay of cAMP Levels in Isolated Cardiac Myocytes

For measurement of receptor generated cAMP accumulation, plated cardiac myocytes were stimulated with of isoproterenol (15 min) in the presence of IBMX. Separated groups were pretreated with 250 ng/ml pertussis toxin (PTX) (Alexis Biochemicals) overnight, before stimulation with isoproterenol. Control experiment of the efficacy of PTX treatment was performed in parallel with stimulation with 10 uM of carbachol (Sigma-Aldrich). The reactions were stopped by rapidly aspirating the medium and adding ml ice-cold 0.1M HCl. The total synthesized cAMP from the myocytes were measured by a radioimmunoassay ([I¹²⁵]-cAMP Flashplate assay, PerkinElmer Life and Analytical Sciences, Inc.) according to the manufacturer's instructions.

Radioligand Binding Assay

The density of β-AR were measured in extracts from myocardial tissue samples from NLC and Tg-CTGF mice in 96-well plates by radioligand binding assay using 0.03-0.07 nM (−)-3-[¹²⁵I]-iodocyanopindolol (Specific activity 2000 Ci/mmol; GE Healthcare, Inc.) with or without 10 mM (−)-propranolol in a binding buffer containing 50 mM Tris-HCl, pH 7.5, 1 mM EDTA, 5 mM EGTA, 2 mM MgCl₂, 1 mM ascorbate, 0.1% BSA and 100 mM GTP. The plates were incubated at 25° C. for 90 min and harvested onto UniFilter-96 GF/C (Packard Instrument Co., Meriden, Conn.). Non-specific binding was defined as binding that was not replaced by excess (−)-propranolol (10 mM).

Analysis of Myocardial Hydroxyproline Contents

Quantitative analysis of tissue contents of hydroxyproline was performed by HPLC using AccQ-Fluor reagent kit (Waters Corporation Milford, Mass., USA). Briefly, cardiac tissue samples (5-6 mg dry weight) were hydrolyzed in 6 M HCl for 16 h at 110° C. The samples were subsequently dried under vacuum, dissolved in 20 mM HCl. In a typical analysis 10-20 ul of sample was added with borate buffer (AccQ-Fluor borate buffer) to yield a total volume of 80 ul. Derivatization was initiated by the addition of 20 ul AccQ-Fluor reagent (3 mg/ml in acetonitrile). The reaction was performed at 55° C. and completed within 10 min. The samples were finally subjected to HPLC-chromatography using a 20×3.9 mm Sentry Guard column (Nova-Pak Cis bonded silica) connected to a 150×3.9 mm AccQ-Tag reversed-phase column (both from Waters). The mobile phase was programmed at a flow of 1.0 ml/min starting with 100% solvent A (140 mM sodium acetate containing 17 mM triethylamine, pH 4.95), followed by a linear gradient to 60% solvent B (acetonitrile in water) for 35 min. Detection was accomplished by fluorescence with excitation at 250 nm and emission at 395 nm. Elution of hydroxyproline from myocardial tissue samples was verified and quantified by co-elution with known amounts of derivatized hydroxyproline standards (Fluka, Buchs SG, Switzerland). The relation of myocardial hydroxyproline contents to myocardial collagen has previously been reported by Laurent et al 1981 Anal Biochem 113(2):301-312

Immunohistochemistry and Histological Analysis

Immunohistochemical analysis of myocardial tissue sections (6 um) was performed using the purified rabbit anti-CTGF IgG as described previously. In addition, monoclonal rat anti-mouse CD34 IgG₂ₐ (Abeam, Cambridge, UK) and polyclonal goat anti-fibronectin IgG (Santa Cruz biotechnology, Inc.) were used for detection of myocardial blood vessels and fibronectin in myocardial tissue sections, respectively. The avidin-biotin-peroxidase system (Vectastain Elite kit, Vector Laboratories, Calif., USA) was used for signal amplification. Non-immune IgG or omission of primary antibody was used as negative controls. Other myocardial tissue sections were stained with Masson's trichrome solution or Periodic Acid-Schiff (PAS) reagents to assess the contents of myocardial extracellular collagen and glycogen, respectively. The specificity of glycogen staining by PAS was confirmed by pretreatment of parallel tissue sections with α-amylase prior to PAS-staining. The contents of glycogen in myocardial tissue sections were assessed by determination of the relative area of PAS staining (purple-stained glycogen) in terms of pixels relative to the total number of pixels in the field of study by analysis of digitally captured visual fields using Photoshop 9.0 (Adobe Systems, San Jose, Calif., USA).

Analysis of Contractility of Isolated Papillary Muscles

Tg-CTGF and NLC mice (male, 6 months) were anesthetized using sodium pentobarbital (10 mg i.p.) and euthanized by excision of the heart. The aorta was cannulated and the heart was subjected to retrograde perfusion with relaxing buffer (118.3 mmol/l NaCl, 3.0 mmol/l KCl, 0.5 mmol/l CaCl₂, 4.0 mmol/l MgSO₄, 2.4 mmol/l KH₂PO₄, 24.9 mmol/l NaHCO₃, 10.0 mmol/l glucose, 2.2 mmol/l mannitol) containing 20 mmol/l 2,3-butanedione monoxime (BDM) and equilibrated with 95% O₂/5% CO₂ to pH 7.4 at 31° C. The posterior left ventricular papillary muscle was ligated at each end, carefully excised and mounted in organ baths, and allowed to adapt for 20 min before BDM was washed out, Ca²⁺ was gradually increased to 1.8 mmol/l, and Mg²⁺ lowered to 1.2 mmol/l. The muscles were field-stimulated with alternating polarity at 1 Hz with impulses of 5 msec duration and current about 20% above individual threshold (10-15 mA, determined in each experiment). The isometrically contracting muscles were stretched to the maximum of their length-tension curve. The force was recorded and analyzed as previously described. After equilibration in the presence of prazosin (0.1 (amol/l), isoproterenol was added directly to the organ baths at increasing concentrations until supramaximal concentration of agonist was obtained with respect to inotropic response. Signal averaged contraction-relaxation cycles were calculated for the different experimental periods and used to determine the inotropic response (dF/dtₘₐₓ) as function of increasing concentrations of isoproterenol.

Transthoracic Echocardiography of Cardiac Dimensions

Transthoracic echocardiography was performed using the Vivid 7 System (GE Vingmed Ultrasound, Horten, Norway) and a 13 MHz linear array transducer. 2D-guided M-mode recordings of the LV in the short-axis view at the level of the papillary muscle were obtained, and interventricular septum and posterior wall thickness at end-diastole (IVST and PWT, respectively) were measured. Internal LV end-diastolic and end-systolic diameters (LVEDD and LVESD, respectively) were recorded as the largest anterio-posterior diameter. All echocardiographic recordings were performed under sedation with midazolam (6.25 mg/kg s.c). All dimensions were analyzed off-line using the EchoPac software analysis program (GE Vingmed Ultrasound, Horten, Norway) by a trained specialist who had no knowledge of the study groups.

Chronic Pressure Overload-Induced Heart Failure

Seven weeks old, weight-matched male Tg-CTGF mice and NLC mice were randomized to either sham operation (SH) or abdominal aortic banding (AB). The aortic-banded mice groups were Tg-CTGF mice (n=9) and NLC-mice (n=9). Sham-operated mice groups were Tg-CTGF mice (n=7) and NLC mice (n=7). Briefly, suprarenal aortic constriction was performed by placing a suture around the abdominal aorta and a 26-gauge blunted needle, which was subsequently removed. End point analyses were performed 12 weeks after surgery.

Chronic β-Adrenergic Receptor Agonist-Induced Cardiomyopathy Model

Male Tg-CTGF and NLC mice (male, 6 months) were randomized to continuous infusion of isoproterenol (Sigma-Aldrich; 150 mg/kg/day s.c.) or vehicle (saline) for 14 days (n=10 mice in each treatment group for both Tg-CTGF and NLC mice) delivered by subcutaneously implanted micro-osmotic pumps (Alzet®, CA). Echocardiography was performed immediately before implantation of the micro-osmotic pumps and after 14 days of continuous administration of isoproterenol.

Analysis of Cardiac Function In Vivo

In vivo cardiac function was determined by simultaneous LV pressure-volume recording as described by Georgakopoulos et al 1998 Am J Physiol 274(4 Pt 2):H1416-1422 by trans-carotic catheterization of the LV of anesthetized Tg-CTGF and NLC (male, 4 months old) mice using a combined pressure-conductance micro-tip catheter (SPR-853, Millar Instruments, Houston, Tex., USA). The mice were anesthetized with isoflurane (1% isoflurane) and pressure-volume parameters were recorded at heart rate stabilized above 450 beats/min.

Isolated Heart Perfusion and Ischemia-Reperfusion Experiments

Weight-matched male Tg-CTGF mice (n=9) and NLC mice (n=9) were anesthetized by intraperitoneal injections of pentobarbital (50 mg/kg) with concomitant heparin (500 IU) injection. Immediately after induction of anesthesia, hearts were excised through a median sternotomy, the aorta cannulated, and Langendorff-perfused with Krebs-Henseleit buffer (saturated with 5% CO2, 95% O2) either at a constant pressure (55 mmHg) or constant coronary flow (2.5 ml/min) The temperature of the heart was maintained at 37° C. through water-jacketed heating. Global ischemia was achieved by clamping the inflow tubing. Left ventricular systolic (LVSP) and end-diastolic (LVEDP) pressures were recorded via a balloon inserted into the left ventricle, while left ventricular developed pressure (LVDP) was calculated (LVDP=LVSP-LVEDP). LVEDP was set to 5 mmHg at the end of the stabilization period. Coronary flow (CF) was continuously measured by a flowmeter (Transonic Systems Inc.). All recordings of LVSP, LVEDP, LVDP, and heart rate (HR) were registered in a computer system (PCLAB, Astra-Zeneca AB, Molndal, Sweden). After 15 min of stabilization the hearts were subjected to 40 min of global ischemia (no flow), followed by 60 min of reperfusion. At the completion of reperfusion, 1 mm thick sections of the hearts were incubated in 1% triphenyl tetrazolium chloride (TTC, Sigma, St. Louis, Mo.) at 37° C. for 20 min, and fixed in 4% paraformaldehyde. Digital images were obtained with a Epson Expression 1680 Pro scanner. Computerized planimetry of infarct size was carried out in Adobe Photoshop 7.0.

Infarct areas from sections of one heart were averaged into one value for statistical analyses. Perfusion of mouse heart in the absence or presence of recombinant human CTGF was performed essentially as described above. Mouse hearts were perfused with Krebs-Henseleit solution in the absence (n=8) or presence (n=8) of recombinant human CTGF (75 nmol/l) for 10 min immediately preceeding induction of ischemia. Recombinant human CTGF was obtained from EMP Genetech, Ingolstadt, Germany. Briefly recombinant human CTGF was expressed in HEK293 cells and recombinant protein secreted into serum-free medium was purified by sequential Heparin-Sepharose affinity chromatography, S-sepharose ion exchange chromatography, and final size-exclusion chromatography.

Statistical Analysis

All values are given as mean±SEM. Between-group variations were assessed by two-tailed un-paired t-test. For multiple comparisons ANOVA was performed and post-hoc analysis with Bonferroni's test was employed unless otherwise stated in the figure legends. P values<0.05 were considered to be statistically significant.

### Example 1

Characterization of CTGF Transgenic Mice

CTGF could be detected by Western blot analysis of myocardial tissue from non-transgenic control mice albeit at extremely low levels (Figure IB). Given this reference point, the Tg-CTGF/6 mice exhibited approximately 70-fold overexpression of myocardial CTGF compared with corresponding NLC mice (FIG. 1C). As shown in figure ID, immunohistochemical analysis demonstrated robust anti-CTGF immunoreactivity restricted to cardiomyocytes in myocardial tissue section of Tg-CTGF mice. The Tg-CTGF mice developed normally with similar body weight as their non-transgenic counterparts.

Tg-CTGF Mice have Smaller Hearts than Non-Transgenic Control Mice.

For characterization of phenotype, all experiments were performed with age- and sex-matched male (4 months old) Tg-CTGF and corresponding NLC mice. Echocardiographic parameters were obtained in sedated mice (midazolam, 6.25 mg/kg s.c), whereas hemodynamic data were collected under isofluran anesthesia in closed-chest preparation. Body weights of Tg-CTGF and NLC mice were not significant different (21.8 g±0.6 vs. 21.2 g±0.4; P<0.05), whereas heart weight and heart-to-body weight ratios of Tg-CTGF mice were significantly decreased as compared with those of non-transgenic littermates (97.4±2.4 mg vs. 109.5±2.9 mg and 4.5 mg/g±0.1 vs. 5.2 mg/g±0.1, respectively; P<0.05) (Table 1). In addition, echocardiography demonstrated that wall thickness, external cardiac diameter and computed left ventricular mass were significantly lower in Tg-CTGF mice confirming smaller cardiac dimensions of transgenic mice, as shown in Table 1.

Tg-CTGF Mice have Unaltered Cardiac Function

As shown in Table 1, Tg-CTGF mice showed no evidence of cardiac dysfunction. Heart rate was slightly higher in Tg-CTGF mice than NLC mice, whereas LV systolic pressure (LVSP) and LV end-diastolic pressure (LVEDP) were similar in transgenic and NLC mice. Consistent with smaller cardiac dimensions, LV end-diastolic volume (LVEDV) was found to be significantly lower in Tg-CTGF mice compared to NLC mice, but LV end-systolic volume (LVESV) was similar in transgenic and NLC mice. Additionally, there was a trend towards decreased cardiac output for the transgenic line but this decrease was not significant. LV contractility, as assessed by LV (dP/dt)ₘₐₓ and LV (dP/d)tₘiₙ was similar in Tg-CTGF mice and NLC mice, and fractional shortening was unaltered between the groups, indicating normal cardiac function in transgenic mice (Table 1).

Tg-CTGF Mice have Slightly Higher Myocardial Contents of Extracellular Collagen

The sustained overexpression of CTGF causes increased myocardial expression of procollagen α1(1) and procollagen α1 (III) mRNA. As shown in FIGS. 2A and B, real-time PCR analysis revealed significant, although not robust, upregulation of myocardial procollagen α1(1) and procollagen α1(III) mRNA in Tg-CTGF mice compared to NLC mice (11.0±1.4 vs. 6.6±0.2 and 9.8±0.8 vs. 5.0±0.1, respectively, relative to 18 S rRNA P<0.05). Moreover, quantification of myocardial hydroxyproline demonstrated increased total myocardial collagen in the Tg-CTGF mice (2.4±0.13 u/mg vs. 1.7±0.1 ug/mg (collagen weight/myocardial dry weight); P<0.05) (FIG. 2C). Thus, the increased expression of procollagen mRNA reflects in similar increase of myocardial hydroxyproline contents indicating increased interstitial collagen. FIG. 2E, upper two panels, shows Masson's trichrome staining of myocardial section from 4-month-old male NLC and Tg-CTGF mice, demonstrating moderate increase of interstitial collagen (arrows) present in tissue of Tg-CTGF mice compared to NLC mice. In addition, immunohistochemical analysis of fibronectin revealed marked increase of interstitial fibronectin staining in myocardial tissue sections of Tg-CTGF mice (FIG. 2E, middle two panels).

Capillary Density Measurement

To assess the angiogenic effect of constitutive CTGF expression we measured vascular density in myocardial tissue of Tg-CTGF (n=6) and non-transgenic control (n=6) mice. Capillary (microvessel) density of myocardial tissue sections of the CTGF transgenic animals was significantly higher than that of the NLC mice (1930±123 vessels/mm² vs. 1405±96 vessels/mm²; P<0.05) (FIG. 2D). Immunohistochemical staining with anti-CD34 antibody was performed to detect endothelial cells (FIG. 2E, bottom two panels).

Altered Gene Expression in Tg-CTGF Mice.

The Affymetrix software algorithm was used to analyze myocardial mRNA levels by DNA microarray and to examine differentially expressed genes in myocardial tissue of Tg-CTGF mice compared with NLC mice. 470 myocardial genes were found to be significantly altered (increased or decreased) in T-g-CTGF hearts vs. NLC hearts. Myocardial genes that displayed more than 50% increase or decrease of mRNA level are shown in Supplement Table. Differentially regulated genes were further organized into groups according to known biological functions: signal transduction, metabolism, transcription regulation, cellular growth, cellular adhesion and/or intracellular support and apoptosis. Real-time quantitative PCR analysis of RNA isolated from myocytes of Tg-CTGF mice vs. NLC mice was performed to validate differential gene expression from DNA microarray analysis (Table 2). Specific gene expression signature of Tg-CTGF mice includes a marked regulation of the genes putatively involved in control of interstitial matrix composition, as well as genes involved in regulation of myocardial growth, and cardioprotection. DNA microarray analyses revealed gene expression pattern consistent with a profibrotic state, with increased expression levels of procollagen α1(I) and procollagen α1(III). Analysis of the RNA isolated from myocytes also demonstrated regulation of several growth factors including epidermal growth factor (EGF), transforming growth factor β₂ (TGF-β₂) and growth differentiation factor 15 (GDF 15). EGF and GDF 15 have been shown to play significant roles in cardiac growth and function. Strikingly, several of the genes increased in Tg-CTGF mice, i.e. heme oxygenase-1 (HMOX-1), hypoxia inducible factor 1a (HIF-1a) and hexokinase-1 (Hk-1) have previously been suggested as putative cardioprotective genes.

Another myocardial mRNA that was found to be substantially upregulated in the gene expression array of transgenic CTGF mice was G protein-coupled receptor kinase-5 (GRK5). Upregulation of cardiac myocyte GRK5 mRNA levels was verified by real-time quantitative PCR of RNA isolated from cardiac myocytes from Tg-CTGF mice versus non-transgenic control mice (FIG. 3). Real-time quantitative PCR analysis of all GRK isoforms expressed in cardiac myocytes revealed selective upregulation of GRK5 mRNA levels, i.e. the mRNA levels of GRK2, GRK3 and GRK6 were unaltered in cardiac myocytes from Tg-CTGF mice versus nontransgenic control mice. CTGF-mediated upregulation of GRK5 in cardiac myocytes was also demonstrated by immunoblot analysis of extracts cardiac myocytes from Tg-CTGF mice versus non-transgenic control mice.

Functional Consequences of Increased GRK5 Levels in Cardiac Myocytes from Tg-CTGF Mice

GRK5 is well documented to catalyze phosphorylation and desensitization of β-adrenergic receptors on cardiac myocytes. Thus, we investigated β-adrenergic agonist (isoproterenol)-stimulated cAMP generation in cardiac myocytes from Tg-CTGF mice versus nontransgenic control mice. In congruence with the increased GRK5 levels in cardiac myocytes from Tg-CTGF mice we found substantial reduction of the efficacy of isoproterenol in these cells compared with cardiac myocytes from non-transgenic control mice (FIG. 4). In order to investigate to what extent the blunted responses to isoproterenol in cardiac myocytes from Tg-CTGF mice would translate into attenuated isoproterenol-stimulated contractility, we performed concentration-effect curves of isoproterenol-stimulated inotropic responses in isolated papillary muscle strips from transgenic CTGF mice versus nontransgenic control mice. Consistent with the isoproterenol-stimulated cAMP synthesis, the isolated papillary muscle strips from Tg-CTGF mice revealed blunted inotropic responses to isoproterenol (FIG. 5A). These blunted responses to isoproterenol were not due to altered downstream responsiveness to cAMP, since muscle strips stimulated with supramaximal concentrations of dibutyryl-cAMP, a membrane permeable cAMP analog, elicited similar inotropic responses in TG-CTGF mice versus non-transgenic control mice (FIG. 5B).

Increased Myocardial GRK5 Protects from Chronic Isoproterenol-Induced Cardiotoxicity

Tg-CTGF mice and corresponding non-transgenic control mice were subjected to chronic exposure to isoproterenol for 14 days. Isoproterenol or vehicle was administered by micro-osmotic pumps as detailed in the Materials and Methods section. As shown in FIG. 6 chronic administration of isoproterenol lead to similar down-regulation of myocardial β-adrenergic receptor densities in Tg-CTGF mice and non-transgenic control mice. Interestingly, isoproterenol elicited significant increase of cardiac mass in non-transgenic mice, whereas the hypertrophic response in Tg-CTGF mice was blunted. Chronic administration of isoproterenol lead to left ventricular dilatation and impaired systolic function in non-transgenic control mice, whereas left-ventricular dimensions and systolic function were preserved in Tg-CTGF mice.

Tg-CTGF Mice Display Preserved Left Ventricular Geometry and Function after Chronic Pressure Overload In Vivo

Serial echocardiographic recording of mice subjected to pressure overload by aortic constriction revealed inhibition of cardiac hypertrophy and preserved cardiac function in Tg-CTGF mice vs. NLC mice 12 weeks after aortic banding. Important parameters of cardiac structure and function such as left ventricular internal end-diastolic diameter and fractional shortening were preserved in Tg-CTGF mice (FIG. 7A). Absolute and indexed heart weights were substantially elevated in NLC-banded mice compared with sham-operated NLC mice. Conversely, increase of cardiac mass was essentially blunted in Tg-CTGF band mice despite similar increase of cardiac pressure after aortic constriction. Consistently, end-point analysis by in vivo pressure-volume analysis displayed lack of dilatation and preserved ejection fraction in Tg-CTGF mice. Importantly, invasive systolic blood pressure was not significantly different between the banded NLC and Tg-CTGF mice (Table 3). Hypertrophy markers like ANP, BNP and α-skeletal actin were also markedly induced in NLC hearts, and attenuated in Tg-CTGF hearts (FIG. 7B).

Tg-CTGF Mice are Protected against Ischemia/Reperfusion Injury

In order to explore the role of CTGF in cardioprotection, isolated hearts from Tg-CTGF mice (n=8) and NLC mice (n=8) were subjected to 40 min of global ischemia followed by 60 min of reperfusion in a Langendorff system. During reperfusion, Tg-CTGF hearts showed improved recovery of LV-function as compared to NLC-hearts, but importantly no differences in heart rate. Consistently, end-point analysis revealed markedly reduced infarct size in Tg-CTGF hearts as compared to NLC hearts (13.9±2.5% vs. 50.6±8.0%, P<0.001) (FIG. 8A). To exclude the possibility of increased coronary flow contributing to cardioprotection in the constant pressure perfusion system, the experiment was repeated using a constant flow model. Similar end-point analysis revealed reproducibility of the cardioprotective effect of CTGF regarding infarct size (16.7±2.3% vs. 65.9±5.2%, P<0.001) (FIG. 8B).

Recombinant Human CTGF Administered Prior to Ischemia/Reperfusion Reduces Infarct Size and Improves Recovery of Contractile Function.

In order to confirm a direct cardioprotective action of CTGF in the heart, mouse hearts were subjected to Langendorff-perfusion ex vivo with Krebs-Henseleit solution in the absence or presence of recombinant human CTGF (75 nmol/l). The hearts were perfused in the absence or presence of recombinant human CTGF for 10 min followed by 40 min of no-flow ischemia, and then reperfusion for 60 min. Reperfusion was performed with Krebs-Henseleit without any additions. As shown in FIG. 9, mouse hearts that received recombinant human CTGF recovered faster during reperfusion, generated significantly higher left ventricular-developed pressure and acquired significantly smaller infarct size than control hearts (infarct size: 26±4% vs. 39±3% of left ventricular transverse sectional area in CTGF-perfused hearts vs. control hearts, respectively; mean±SEM of n=8 in each group; P<0.05).

Activation of the Smad2 and Akt/GSK-3β Signalling Pathways in Tg-CTGF Heart

The Smad2/3-dependent pathway has been reported to confer both antihypertrophic and antiapoptotic effects in the heart. Western blot analysis of myocardial extracts revealed that levels of Smad2 phosphorylation at serine (Ser 465/467) were significantly elevated (4-fold, P<0.05) in the transgenic heart compared with wild-type heart (FIG. 10A), whereas phospho-smad3 levels remained unchanged between the groups (data not shown). It has been reported that many noxious stimuli, including ischemia/reperfusion activate several protein kinases, including Akt/protein kinase B²⁸ and that activation of Akt induces survival of cells. Protection of Tg-CTGF hearts against ischemia/reperfusion injury prompted us to investigate the activation status of Akt by Western blotting with a phospho-specific antibody. We observed a marked increase in Akt phosphorylation at serine-473 in transgenic hearts compared with control hearts, but absolute protein levels were unaltered (FIG. 10B). GSK-3β is a kinase with profound effects on fetal development and tumorigenesis. The activity of GSK-3β is negatively regulated by Akt in many cell types, and inhibition of GSK-3β by phosphorylation seems to be critical to the anti-apoptotic effects of Akt. Consistent with the activation of Akt, we observed a significant phosphorylation of GSK-3β at serine-9 in transgenic hearts (FIG. 10C). Phosphorylation of GSK-3β which causes inhibition of GSK-3β consequently increases the activity of glycogen synthase (GS). We next examined the phosphorylation status of GS. The level of GS phosphorylation at serine-641 in transgenic mice was substantially lower than that in NLC mice, suggesting increased GS activity in transgenic hearts (FIG. 10D).

Increased Myocardial Glycogen Content in Tg-CTGF Mice

Phosphorylation (Ser9) of GSK-3P causes inhibition of GSK-3P resulting in decrease of GS phosphorylation and consequently increased activity of GS. To examine whether inhibition of GSK-3P in transgenic heart resulted in increased storage of myocardial glycogen, we performed histochemical staining of myocardial tissue sections from transgenic mice (n=8) and NLC mice (n=7). FIG. 10E shows PAS staining of hearts from transgenic and NLC mice (top two panels). Pretreatment with a-amylase confirmed the specificity of PAS staining (bottom two panels) Analysis of myocardial contents of glycogen in the tissue sections by, determination of the relative area of purple-stained glycogen in multiple visual fields of each section revealed significant increase in myocardial glycogen contents in transgenic mice compared to NLC mice (6.2±0.5% vs. 2.7±0.2%, /><0.001) (FIG. 10F).

TABLE 1
Cardiac mass, pressure-volume analysis and echocardiographic
parameters of male age-matched Tg-CTGF and non-
transgenic control mice (NLC) mice

| | NLC | Tg-CTGF |
|---|---|---|
| Cardiac mass | n = 10 | n = 10 |
| Body weight (g) | 21.2 ± 0.4 | 21.8 ± 0.6 |
| Heart weight (wet, mg) | 109.5 ± 2.9 | 97.4 ± 2.4* |
| Heart weight (dry, mg) | 22.5 ± 0.4 | 19.2 ± 0.3* |
| Heart weight/Body weight (mg/g) | 5.2 ± 0.1 | 4.5 ± 0.1* |
| Pressure- volume analysis | n = 8 | n = 7 |
| HR (bpm) | 517 ± 18.6 | 524 ± 13.6 |
| LVSP (mmHg) | 100.0 ± 2.2 | 98.5 ± 3.2 |
| LVEDP (mmHg) | 1.1 ± 1.9 | 1.0 ± 1.5 |
| LVESV (μl) | 12.3 ± 1.5 | 10.0 ± 0.4 |
| LVEDV (μl) | 21.8 ± 1.7 | 16.8 ± 1.3* |
| SV (μl) | 13.9 ± 0.8 | 10.9 ± 1.0 |
| SW (mmHg/μl) | 1167 ± 90 | 951 ± 126 |
| EF % | 58.0 ± 2.6 | 55.4 ± 3.4 |
| LV +dp/dt (mmHg/s) | 10970 ± 710 | 10290 ± 738 |
| LV −dp/dt (mmHg/s) | 8879 ± 807 | 8874 ± 949 |
| Cardiac output (μl/min) | 7211 ± 394 | 5820 ± 555 |
| Tau g (ms) | 11.5 ± 1.2 | 11.7 ± 0.8 |
| Tau w (ms) | 5.0 ± 0.4 | 5.0 ± 0.4 |
| Echocardiographic measurements | n = 24 | n = 25 |
| IVSDD (mm) | 0.97 ± 0.02 | 0.87 ± 0.02* |
| LVEDD (mm) | 2.16 ± 0.05 | 2.03 ± 0.07 |
| LVSD (mm) | 1.18 ± 0.05 | 1.10 ± 0.05 |
| PWEDD (mm) | 0.98 ± 0.02 | 0.94 ± 0.03 |
| FS (%) | 45.1 ± 1.4 | 45.6 ± 1.2 |
| RWT | 0.91 ± 0.03 | 0.88 ± 0.04 |
| External Cardiac Diameter (mm) | 4.11 ± 0.06 | 3.84 ± 0.07* |
| LV Mass (mg) | 72.0 ± 3.0 | 60.0 ± 3.0* |

TABLE 2
Quantitative realtime PCR analysis of myocardial gene expression of Tg-CTGF and NLC mice

| | mRNA/18S rRNA | (Relative Units) | |
|---|---|---|---|
| Gene Name | NLC | Tg-CTGF | Fold Change |
| Epidermal growth factor | 2.05 ± 0.40 | 0.91 ± 0.26 | ↓ 2.0 |
| Growth differentiation factor 15 | 1.00 ± 0.11 | 9.10 ± 2.00 | ↑ 9.0 |
| Transforming growth factor β2 | 0.18 ± 0.01 | 0.48 ± 0.09 | ↑ 2.6 |
| Activating transcription factor 4 | 0.95 ± 0.07 | 3.39 ± 0.58 | ↑ 3.6 |
| Activating transcription factor 5 | 0.21 ± 0.01 | 1.86 ± 0.48 | ↑ 9.0 |
| Cyclin-dependent kinase inhibitor 1A (P21) | 0.95 ± 0.15 | 5.64 ± 1.27 | ↑ 6.0 |
| Cathepsin L | 0.73 ± 0.05 | 1.51 ± 0.18 | ↑ 2.0 |
| Procollagen-proline, 2-oxoglutarate 4-dioxygenase α1 | 0.99 ± 0.06 | 1.90 ± 0.29 | ↑ 2.0 |
| Heme oxygenase (decycling) 1 | 1.48 ± 0.26 | 6.25 ± 1.41 | ↑ 4.2 |
| Hexokinase 1 | 0.87 ± 0.02 | 2.15 ± 0.40 | ↑ 2.5 |
| Hypoxia inducible factor 1, alpha | 5.03 ± 0.18 | 7.48 ± 0.76 | ↑ 1.5 |
| Phosphoglycerate kinase 1 | 0.84 ± 0.12 | 1.34 ± 0.16 | ↑ 1.6 |

TABLE 3
Cardiac mass and pressure volume analysis of male age-matched
Tg-CTGF and NLC mice 12 weeks after aortic banding

| | NLC sham | Tg-CTGF sham | NLC band | Tg-CTGF band |
|---|---|---|---|---|
| | n = 6 | n = 7 | n = 9 | n = 9 |
| Cardiac mass | | | | |
| Body weight (g) | 33.9 ± 0.9 | 31.6 ± 0.7 | 34.7 ± 0.7 | 34. ± 1.6 |
| Heart weight (mg) | 168.3 ± 7.1 | 161.9 ± 3.3 | 245.7 ± 19.3† | 156.7 ± 3.5* |
| LV weight (mg) | 146.5 ± 11.1 | 128.0 ± 2.0 | 200.3 ± 15.8† | 120.3 ± 3.76* |
| Tibia length (cm) | 1.93 ± 0.03 | 1.95 ± 0.01 | 1.98 ± 0.02 | 1.97 ± 0.01 |
| Heart weight/tibia length (mg/cm) | 95.7 ± 6.7 | 83.1 ± 2.3 | 125.1 ± 8.8† | 79.7 ± 2.1* |
| LV weight/tibia length (mg/cm) | 76.1 ± 5.6 | 66.0 ± 1.0 | 101.1 ± 7.1† | 60.9 ± 2.1* |
| Pressure volume analysis | | | | |
| HR (bpm) | 586 ± 22 | 602 ± 11 | 647 ± 19 | 639 ± 17 |
| Systolic blood pressure (mmHg) | 94.6 ± 3.0 | 90.0 ± 4.5 | 120.8 ± 6.8† | 115.3 ± 2.6† |
| LVESP (mmHg) | 90.5 ± 3.8 | 95.4 ± 3.8 | 154.0 ± 9.5† | 106.7 ± 3.19†* |
| LVEDP (mmHg) | 3.1 ± 1.6 | 2.5 ± 2.3 | 11.6 ± 2.1† | 6.7 ± 0.8* |
| LVESV (μl) | 9.6 ± 1.2 | 6.0 ± 1.5 | 19.4 ± 3.7† | 7.0 ± 1.6* |
| LVEDV (μl) | 18.9 ± 1.9 | 19.8 ± 0.9 | 27.8 ± 2.5† | 20.3 ± 1.8* |
| Cardiac output (μl/min) | 9532 ± 614 | 9215 ± 674 | 8079 ± 887 | 9262 ± 516 |
| EF (%) | 66 ± 6 | 75 ± 5 | 45 ± 7† | 71 ± 5* |
| Elastance (mmHg/μl) | 8.0 ± 1.3 | 6.4 ± 0.6 | 13.7 ± 2.6 | 7.5 ± 0.3* |

### Summary

This first report on the function of myocardial CCN2/CTGF reveals novel and unexpected actions of CTGF in the heart. In summary, although CTGF elicits increased myocardial procollagen α(I) I and III mRNA levels and subtle increase of myocardial collagen, myocardial interstitial fibrosis was inconspicuous. More readily discernable, CTGF caused inhibition of cardiac growth both under physiological conditions as well as under chronic pressure overload. Indeed, Tg-CTGF mice displayed remarkable resistance to dilated cardiomyopathy and heart failure compared with their corresponding non-transgenic littermates (NLC). Even more notable was the increased tolerance of hearts from Tg-CTGF mice as well as of hearts perfused with recombinant CTGF to ischemia-reperfusion injury. Thus, the current study demonstrates that CTGF is an endogenous cardioprotective factor that may halt onset of heart failure or reduce myocardial infarction following ischemia-reperfusion injury of the heart.

The present study shows that transgenic mice overexpressing CTGF under control of the cardiac myocyte-specific α-myosin heavy chain promoter, display a distinctive cardiac phenotype consistent with the minimal leakage of the α-MHC promoter. In this respect, Tg-α-MHC-CTGF mice had a similar body mass to their non-transgenic littermate control mice. Yet, cardiac mass was significantly reduced in Tg-CTGF mice compared with NLC mice. Thus, expression of CTGF in the postnatal heart inhibits cardiac growth, a finding that also reflected in smaller dimensions of the cardiac myocytes. The decreased cardiac mass of Tg-CTGF mice vs. NLC mice is also reflected in decreased dimensions of the heart, i.e. the left ventricular end-diastolic diameter and left ventricular wall thickness. However, cardiac contractility was not impaired, indicating that the slight decrease of myocardial mass did not affect cardiac contractility under physiological conditions.

As reported in this study, CTGF apparently induces both procollagen α (I) mRNA expression as well as myocardial collagen contents. Yet, the 20%-increase of myocardial collagen contents in the Tg-CTGF mice is marginal compared with the 70-fold overexpression of myocardial CTGF levels in the same mice. Consistently, the minor increase of myocardial collagen neither affected the diastolic nor the systolic functions of the heart as determined by simultaneous in vivo pressure-volume analysis. Thus, initiation of myocardial fibrosis does not appear to an important function of CTGF in the heart. At the least, additional cofactors appear to be necessary for CTGF to express potent profibrotic properties.

The myocardial gene expression signatures of Tg-CTGF mice also supported the profibrotic properties and the growth-inhibitory/anti-hypertrophic actions of CCN2/CTGF. As far as extracellular matrix proteins are concerned, analysis of global myocardial gene expression in Tg-CTGF mice versus NLC mice revealed increased expression of procollagen α(I) and α(III), TGF-β2, and fibrillin mRNAs. Similarly, among the more substantially regulated genes, increased myocardial mRNA expression of the TGF-β family members TGF-β2 and GDF-15, increased myocardial p21 mRNA levels, and reduced myocardial mRNA levels of EGF, collectively support the growth inhibitory actions of CTGF. Although the individual contribution of these regulated genes to the phenotype of Tg-CTGF mice cannot readily be determined, gene targeting or cardiomyocyte-restricted overexpression of these gene have clearly assigned a role for any one of them in regulation of myocardial growth consistent with the proposed action in Tg-CTGF mice. However, profiling of myocardial gene expression in Tg-CTGF mice also provided clues of activation of another gene program that might elicit secondary inhibition of myocardial growth. This gene program comprised the gene expression signature of the unfolded protein response. Prototypical genes of this gene program, for example activating transcription factor-4 and -5, and asparagines synthetase, were substantially induced in cardiac myocytes from Tg-CTGF mice. Activation of the unfolded protein response also turns off protein translation with subsequent inhibition of cellular growth. Increased myocardial expression of unfolded protein response genes as well as of genes encoding scavengers of free radicals, for example heme oxygenase-2 and methylene tetrahydrofolate dehydrogenase led us hypothesize that CTGF may act as a as cardioprotective factor. As demonstrated in this report Tg-CTGF mice demonstrated remarkable resistance to ischemia-reperfusion injury. Thus, we contend that the mechanisms of the cardioprotective effect of CTGF are through activation of cardioprotective signaling pathways including activation of a cardioprotective gene program and not increased tissue perfusion. Although subtle increase of capillary density was detected by anti-CD34 immunostaining, the relevance of this finding is dubious, since cardiac myocyte dimensions were also decreased. Furthermore, the cardioprotective effects of CTGF were quantitatively similar irrespective of perfusion of the heart at constant pressure or at constant flow conditions. Thus, the cardioprotective effects of CTGF do not appear to be related to putative differences in regional myocardial blood flow, but rather a direct autocrine/paracrine of CTGF at cardiac myocytes.

Although the intracellular signaling pathways of CTGF are poorly characterized, myocardial tissue of Tg-CTGF mice revealed increased levels of phospho-SMAD2(Ser465/467), phospho-AKT(Ser473) and phospho-GSK-3β(Ser9). Incidental reports of CTGF-stimulated phosphorylation of SMAD2, AKT and GSK-3β in renal mesangial do exist. Interestingly, phospho-SMAD2(Ser465/467) and phospho-AKT(Ser473) activities, as well as inhibition of GSK-3β through phosphorylation of serine-9, have all been reported to confer cardioprotection. Although AKT may phosphorylate GSK-3β at serine-9 with subsequent inhibition of GRK-3β, other kinase cascades may also phosphorylate GSK-3β at this serine residue. Thus, several signaling pathways may converge on GSK-3β, a reported common mediator of cardioprotection against ischemia-reperfusion injury. One of the best characterized substrates of GSK-3β, glycogen synthase displayed decreased phosphorylation at serine 641, consistent with inhibition of GSK-3β in the Tg-CTGF mice. As phosphorylation levels of glycogen synthase are decreased, inhibition is relieved and synthase activity increases with subsequent accumulation of glycogen. Consistently, myocardial tissue of Tg-CTGF mice revealed increased contents of glycogen versus that NLC of mice. Although, the mechanisms of the cardioprotective actions of GSK-3β inhibition are somewhat elusive, the data in the present report provide strong, consistent evidence of inhibition of GSK-3β. Inhibition of GSK-3β, however, may not entirely account for the increased tolerance towards ischemia/reperfusion injury. SMAD2 signaling is also reported to confer cardioprotection and apparently does not crosstalk with GSK-3β. Thus, at least part of the gene expression signature of CTGF may be mediated via the SMAD2/SMAD4 signalling pathway. Activation of SMAD2 also confers antihypertrophic signaling to cardiac myocytes, an explicit finding of Tg-CTGF mice. In this respect the mechanisms that confer cardioprotection against ischemia/reperfusion injury may also protect from aortic banding-induced cardiac dysfunction and heart failure. Conceivably, antihypertophic signaling, scavenging of free oxygen radicals, as well as activation of unfolded protein response genes may all protect from cardiac dysfunction following chronic cardiac stress.

Although the quantitative contribution to cardioprotection played by increased myocardial GRK5 levels in the Tg-CTGF mice is yet to be determined, GRK5 has several functions that conceivably could also confer cardioprotective properties. GRK5 is known to phosphorylate and desensitize cardiac β-adrenergic receptor as well as AT₁ angiotensin receptors. Both receptors are established targets for treatment of chronic heart failure (β-blockers and AT₁ receptor antagonists). Furthermore, recent findings show that GRK5 may not only desensitize and uncouple these receptor from G protein signalling, but indeed initiate ERK1/2 signalling through recruitment of β-arrestin. In fact, certain β-blockers in clinical use (eg. carvedilol) may act as a biased ligand both blocking β-adrenergic receptor activation of G protein signalling and activating the extracellular signal-regulated kinase ERK1/2. ERK1/2 has been shown to confer cardioprotective actions in the heart both in ischemia/reperfusion injury and in chronic heart failure. Thus, increased GRK5 activities in the Tg-CTGF mice is a tantalizing finding and suggest that CTGF may confer cardioprotection through several pathways including both the phosphokinase pathways and regulation of gene expression. Recently, it was reported that a single nucleotide polymorphism of the GRK5 gene in humans resulted in a GRK5 isoform with enhanced GRK5 activities. Interestingly, Chronic heart failure patients with the allele resulting in the more active GRK5 isoform had reduced mortality and improved response to β-blocker therapy.

In conclusion, this study provides new knowledge on the role of CTGF in cardiac physiology and pathophysiology, i.e. novel functions not alluded to by the original denomination as a connective tissue growth factor. In the postnatal heart CTGF protects from ischemia/reperfusion injury and dilated cardiomyopathy following chronic pressure overload by induction of a gene expression signature that includes activation of the unfolded protein response, scavenging of free oxygen radicals, inhibition of myocardial hypertrophy, as well as inhibition of G Protein signalling though β-adrenergic receptors and AT₁ angiotensin receptors. In addition, activation of the phosphoproteome via phospho-SMAD2 signalling and inhibition of GSK-3β signalling is also an important mechanism of the cardioprotection offered by CTGF. Thus, current data open the promise of CTGF as a novel pharmacologic modality to protect from myocardial infarction in unstable angina and acute coronary syndromes.

### Example 2

Recombinant Human CTGF Activates the Cytoprotective Akt/GSK-3β Phosphokinase Signalling

As shown in FIG. 13, cardiac myocytes stimulated with purified recombinant CTGF for 30 min at 37° C. demonstrated concentration dependent increase of phospho-Akt(Serine473) and phospho-GSK-3β(Serine9) levels. Cells were incubated with CTGF and subsequently harvested in RIPA buffer in the presence of phosphatase inhibitor (sodium orthovanadate and sodium fluoride), denatured in Laemmli buffer and subjected to polyacrylamide gel electrophoresis. Proteins were transferred by electroblotting onto PVDF membranes and subjected to immunostaining with anti-phospho(ser473)-Akt and anti-phospho(ser9)-GSK-3β—specific antibodies and secondary HRP-conjugated anti-rabbit IgG, as described above.

As shown in FIG. 14, cardiac myocytes stimulated in the presence or absence of phosphoinositide-3 kinase (PI3K) inhibitor LY294002 or Akt-selective inhibitor API-2 demonstrate that CTGF stimulates the Akt/GSK-3β signaling pathway. Cardiac myocytes were stimulated with recombinant human CTGF (200 μmol/L) for 30 min at 37° C. in the presence or absence of phosphoinositide-3 kinase inhibitor (LY294002; 50 μmol/L) or Akt-inhibitor (API-2; 10 μmol/L). Cells were subsequently harvested in RIPA buffer in the presence of phosphatase inhibitor (sodium orthovanadate and sodium fluoride), denatured in Laemmli buffer and subjected to polyacrylamide gel electrophoresis. Proteins were transferred by electroblotting onto PVDF membranes and subjected to immunostaining with anti-phospho(ser9)-GSK-3β—specific antibody and secondary HRP-conjugated anti-rabbit IgG.

FIG. 15 shows that tolerance to ischemia/reperfusion injury in hearts from Tg-CTGF mice is sensitive to PI3-kinase inhibitor LY-294002. In this study Langendorff-perfused hearts from Tg-CTGF or wil type mice were subjected to cycle of ischemia (40 min) and reperfusion (60 min). Hearts from wild-type and Tg-CTGF mice subjected to perfusion ex vivo in Krebs-Henseleit buffer ad modum Langendorff. Tg-CTGF mice were pretreated with phosphoinositide inhibitor LY294002 (15 μmol/kg/day s.c.; n=6 or DMSO vehicle; n=6) for 5 days to inhibit CTGF-stimulated, PI3 kinase-dependent phosphorylation and activation of Akt and compared with hearts from non-transgenic control mice (NLC; n=6). Hearts were subjected to 40 min of no-flow ischemia and subsequent reperfusion for 60 min in Krebs Henseleit buffer. Infarct size were subsequently assessed by 2,3,5-triphenyltetrazolium chloride (TTC)-staining of remaining viable tissue in serial segments of the left ventricle.

Recombinant Human CTGF Protects Against Ischemia/Reperfusion Injury when Administered after Ischemia

Data shown in FIG. 16 demonstrate that hearts exposed to recombinant CTGF/CCN2 in reperfusion buffer display significantly smaller area of myocardial infarction than heart exposed to reperfusion buffer without CTGF/CCN2. C57/BL6 (wild type) mice were subjected to Langendorff-perfusion in Krebs Henseleit buffer ex vivo. Hearts were subjected to 40 min of global ischemia and subsequently reperfusion in Krebs Henseleit buffer in the presence (n=6) or absence (n=6) of recombinant human CTGF (100 nmol/L). Hearts were exposed to CTGF during the first 10 min of reperfusion. Reperfusion were continued in Krebs Henseleit buffer for a total of 60 min before experiment were terminated and myocardial infarct size were determined by TTC-staining of serial segments of the left ventricle.

### Example 3

Patients Responding with Increasing Plasma CTGF Levels after Acute Coronary Syndromes and Percutanenous Coronary Intervention (PCI) Display Reduced Infarct Size, Reduced End-Diastolic and End-Systolic Volume Index, and Increased Ejection Fraction Compared with Patients with Decreasing Plasma CTGF Levels.

Patients in this study were admitted to hospital for acute myocardial infarction and subjected to PCI (n=50) for revascularization. The patients were observed for 12 months after the event. Functional MRI was performed 2 days after MI and subsequently at 2 weeks, 2 months and 1 year after the acute event. Simultaneously, blood samples were drawn to investigate plasma CTGF levels, CRP and troponin T levels.

Plasma levels of CTGF (mean values of group) were not statistically different at any of the time points (FIG. 17). However, the course of plasma CTGF levels after the index event correlated with infarct size and cardiac function. When patients were stratified according to the course of plasma CTGF after myocardial infarction, it was revealed that patients displaying increased plasma CTGF levels during the first 2 months after MI had significantly smaller infarct size and improved cardiac function compared to patients with decreasing plasma CTGF levels after the index event.

Functional MRI performed at day 2, 14 and 2 months and 1 year after the index event revealed that increased plasma CTGF levels were associated with reduced left ventricular volumes and increased ejection fraction. In other words, patients generating increased plasma CTGF levels after MI had preserved cardiac function compared with patients that did not mount a rise in CTGF levels. Plasma Troponin levels and infarct size determined by MRI (gadolinium contrast) were consistent in demonstrating reduced myocardial infarction in patient with increasing plasma CTGF levels after the ischemic event versus patients with decreasing plasma CTGF levels after the ischemic damage.

Plasma CTGF levels were determined by EIA using monoclonal anti-CTGF IgG₁ capture antibody and anti-CTGF biotin-conjugated (rabbit IgG) detection antibody obtained from R&D Systems Europe Ltd (Abingdon, United Kingdom).

These data are consistent with data from Tg-CTGF mice and from hearts perfused with recombinant human CTGF demonstrating cardioprotective action of CTGF.

1. Connective Tissue Growth Factor (CTGF) for use in the treatment of a subject who has incurred or is incurring damage to the heart, wherein said CTGF is for administration during or after the heart-damaging event.

2. (canceled)

3. The CTGF of claim 1, wherein the CTGF or the medicament is for acute administration.

4. The CTGF of claim 1, wherein said damage is myocardial damage.

5. The CTGF of claim 1, wherein said damage arises from ischaemia, ischaemia/reperfusion injury, hypoxia, increased cardiac workload or cardiac stress, increased pressure on the heart, a cardiotoxic substance, infection, or a maladaptive response of the heart to injury or disease.

6. The CTGF of claim 1, wherein the subject has undergone or is undergoing an ischaemic event.

7. The CTGF of claim 1 for use in the treatment of an acute coronary syndrome.

8. The CTGF of claim 7, wherein the CTGF is for administration during or after the acute coronary syndrome.

9. The CTGF of claim 8, wherein the CTGF is for administration immediately after the acute coronary syndrome.

10. The CTGF of claim 7, wherein the acute coronary syndrome is myocardial infarction.

11. The CTGF of claim 1, wherein said CTGF reduces or ameliorates the heart damage, or protects the heart from damage during or from the acute coronary syndrome or ischaemic event.

12. The CTGF of claim 1, wherein infarct size is reduced.

13. The CTGF of claim 1, wherein cardiac function is improved.

14. The CTGF of claim 1, wherein the CTGF is used
(i) to prevent or delay the onset or development of heart failure after myocardial infarction (MI); or
(ii) to prevent or reduce the extent of MI; or
(iii) before, during or after percutaneous coronary intervention (PCI).

15. The CTGF of claim 14, wherein the CTGF is for administration after an acute coronary syndrome but before, during or after restoration of coronary blood flow, preferably immediately before restoration of coronary blood flow or immediately after re-opening of thrombotic blood vessels, or during PCI.

16. The CTGF of claim 1 for use in the treatment of acute heart failure.

17. The CTGF of claim 1 for use in the treatment of chronic heart failure.

18. Connective Tissue Growth Factor (CTGF) for use in protection of the heart during or after surgery, wherein the CTGF is for administration to a subject immediately before, or during or after surgery.

19. (canceled)

20. (canceled)

21. The CTGF of claim 1, wherein the CTGF is a polypeptide comprising all or part of the amino acid sequence of SEQ ID NO. 1 or an amino acid sequence having at least 50% sequence identity thereto.

22. A method of treatment of a subject who has incurred or is incurring damage to the heart, said method comprising the administration of Connective Tissue Growth Factor (CTGF) to said subject, wherein said CTGF is administered during or after the heart-damaging event.

23. A method of protecting the heart during or after surgery, said method comprising administering Connective Tissue Growth Factor (CTGF) to a subject immediately before, during or after surgery.

24. A pharmaceutical composition comprising Connective Tissue Growth Factor (CTGF) together with at least one pharmaceutically acceptable carrier, diluent or excipient, wherein said composition is for use in
(i) the treatment of a subject who has incurred or is incurring damage to the heart, wherein said CTGF is for administration during or after the heart-damaging event, or
(ii) protection of the heart during or after surgery, wherein the CTGF is for administration to a subject immediately before, or during or after surgery.

25. A medium for storage or transportation of an isolated heart, said medium comprising Connective Tissue Growth Factor (CTGF).


SEQUENCE LISTING (9 sequences)

SEQ ID NO: 1 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 2 - protein, 347 aa, Rattus norvegicus
MLASVAGPVSLALVLLLCTRPATGQDCSAQCQCAAEAAPRCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTVVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 3 - protein, 348 aa, Mus musculus
MLASVAGPISLALVLLALCTRPAMGQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 4 - DNA, 2334 bp, Homo sapiens
gctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

SEQ ID NO: 5 - protein, 16 aa, Artificial sequence
  CTGF fragment
IRTPKISKPIKFELSG

SEQ ID NO: 6 - protein, 6 aa, Artificial sequence
  CTGF fragment
AYRLED

SEQ ID NO: 7 - DNA, 23 bp, Artificial sequence
  primer
cagtggtggaatgcctttaatga

SEQ ID NO: 8 - DNA, 28 bp, Artificial sequence
  primer
aggagtagaatgttgagagtcagcagta

SEQ ID NO: 9 - DNA, 20 bp, Artificial sequence
  probe
ctcagaagaaatgccatcta